(19) United States
(12) Patent Application Publication
(10) Pub. No.: US 2022/0226268 A1
(43) Pub. Date: Jul. 21, 2022

(54) COMPOSITIONS AND METHODS FOR TREATING CENTRAL NERVOUS SYSTEM DISORDERS

(71) Applicant: PaxMedica, Inc., Tarrytown, NY (US)

(72) Inventors: Hock Seng Tan, East Brunswick, NJ (US); Michael Derby, Greenwich, CT (US); Zachary Rome, Nyack, NY (US)

(73) Assignee: PaxMedica, Inc., Tarrytown, NY (US)

(21) Appl. No.: 17/615,673

(22) PCT Filed: May 2, 2020
(86) PCT No.: PCT/US20/31217

§ 371 Date: Dec. 1, 2021

Related U.S. Application Data
(60) Provisional application No. 62/858,621, filed on Jun. 7, 2019.

(51) Int. Cl. A61K 31/185; A61K 47/40; A61K 47/18; A61K 47/38; A61K 47/12; A61K 47/02; A61K 9/00; A61K 9/08

(52) U.S. Cl. CPC A61K 31/185; A61K 47/40; A61K 47/186; A61K 9/08; A61K 47/12; A61K 47/02; A61K 9/0043; A61K 47/38

(57) ABSTRACT

The present invention provides compositions and methods for treating cognitive, social, or behavioral disabilities, and neurodevelopmental disorders such as autism spectrum disorder (ASD) and other central nervous system disorders such as fragile X syndrome (FXS), fragile X-associated tremor/ataxia syndrome (FXTAS), chronic fatigue syndrome (CFS), and post-traumatic stress syndrome (PTSD). The present invention provides compositions and methods for the intranasal delivery (IN) of a therapeutically effective amount of an antipurinergic agent such as suramin for treating the disorder in a patient thereof.

### CROSS-REFERENCE TO RELATED APPLICATIONS

This patent application is a national phase entry under 35 U.S.C. § 371 of International Application Number PCT/US2020/031217, filed May 2, 2020, which claims a benefit under 35 U.S.C. § 119(e) to the Jun. 7, 2019 filing date of U.S. Provisional Patent Application Ser. No. 62/858,621, all of which are herein incorporated by reference-in their entirety.

### FIELD OF THE INVENTION

The present invention provides compositions and methods for treating cognitive, social, or behavioral disabilities, and neurodevelopmental disorders such as autism spectrum disorder (ASD) and other central nervous system disorders such as fragile X syndrome (FXS), fragile X-associated tremor/ataxia syndrome (FXTAS), chronic fatigue syndrome (CFS), and post-traumatic stress syndrome (PTSD). The present invention provides compositions for delivering a therapeutically effective amount of an antipurinergic agent, for example suramin, and pharmaceutically acceptable salts, esters, solvates, and prodrugs of these agents. The agent is delivered by intranasal (IN) administration

### BACKGROUND OF THE INVENTION

Autism is associated with a combination of genetic and environmental factors and has been reported to have an incidence in the US of about 1 in 60 children. Global estimates for autism are about 25 million individuals. Autism is also referred to as autism spectrum disorder (ASD), because it includes a broad range of conditions characterized by challenges with social skills, repetitive behaviors, speech and nonverbal communication. In 2013, the American Psychiatric Association merged four distinct autism diagnoses into the single diagnosis of autism spectrum disorder. These diagnoses include autistic disorder, childhood disintegrative disorder, pervasive developmental disorder-not otherwise specified (PDD-NOS), and Asperger syndrome. Signs of autism usually appear by age 2 or 3. Autism spectrum disorder is a condition related to brain development that impacts how a person perceives and socializes with others, causing problems in social interaction and communication. The disorder can also include limited and repetitive patterns of behavior.

Research shows that early intervention can lead to positive outcomes. See, Chaste P, Leboyer M (2012). “Autism risk factors: genes, environment, and gene-environment interactions”. Dialogues in Clinical Neuroscience. 14 (3): 281-92. PMC 3513682. PMID 23226953; and Centers for Disease Control and Prevention Morbidity and Mortality Weekly Report, Prevalence of Autism Spectrum Disorder Among Children Aged 8 Years—Autism and Developmental Disabilities Monitoring Network, 11 Sites, United States, 2014 Surveillance Summaries/Apr. 27, 2018/67(6); 1-23.

There is currently no cure for autism spectrum disorder, and no US FDA approved medications to treat the core symptoms. Instead what is done is to treat some of the accompanying non-core symptoms with various drugs such as antipsychotics. Symptoms that are often manifested include depression, seizures, anxiety, sleep disorders, and trouble focusing. Also, behavioral therapies and other pharmacological interventions are employed. However, the exact causes of autism are not fully understood, which makes new drug development challenging.

Fragile X syndrome (FXS) is a rare, genetic neurodevelopmental disorder that affects approximately 1 in 4,000 males and females in the US. It is associated with highly variable cognitive and behavioral manifestations and has many overlapping features with ASD. It is an X-linked disorder, meaning that the genetic mutation occurs on the X chromosome. In FXS, there is a trinucleotide repeat expansion in the FMR1 gene. A trinucleotide expansion is a particular type of gene mutation in which a sequence of three nucleotide base pairs improperly repeats itself multiple times. In the case of FXS, the repeating trinucleotide sequence is cytosine-guanine-guanine (CGG). Normally, this DNA segment is repeated from 5 to about 40 times. In people with FXS, the segment is repeated more than 200 times. This typically results in no functional FMR1 mRNA transcript being produced, and the protein that is normally encoded by this transcript (fragile X mental retardation protein (FMRP)) is also absent.

Fragile X-associated tremor/Ataxia (FXTAS) is a different disorder, but genetically related to FXS. It is an “adult onset” rare, genetic neurodegenerative disorder, usually affecting males over 50 years of age. Females comprise only a small part of the FXTAS population, and their symptoms tend to be less severe. FXTAS affects the neurologic system and progresses at varying rates in different individuals.

FXS patients have the “full mutation” in the FMR1 gene (typically well over 200 CGG trinucleotide repeats), but patients with FXTAS are considered premutation ‘carriers’ of the FMR1 gene, as they have CGG trinucleotide repeats numbering in the range of 55-200. The job of the FMR1 gene is to make protein (FMRP) that is important in brain development. Researchers believe that (for unknown reasons) having the premutation leads to the overproduction of FMR1 mRNA (which contains the expanded repeats). Researchers also suspect that the high levels of mRNA are what cause the signs and symptoms of FXTAS, but more research is needed to confirm these hypotheses.

Individuals with FXTAS usually experience symptoms after the age of 55. As premutation carriers age, especially men, the likelihood of experiencing symptoms rises. This likelihood reaches 75 percent by age 75 for premutation men. The progression of symptoms, including memory loss, slowed speech, tremors, and a shuffling gait, is gradual, with interference of daily activities by tremors and falls occurring around ten years after onset of the first symptoms. Dependence on a cane or walker occurs approximately 15 years after first exhibiting the symptoms of the disorder. Some people with FXTAS show a step-wise progression (i.e., symptoms plateau for a period of time but then suddenly get worse) with acute illnesses, major surgery, or other major life stressors causing symptoms to worsen more quickly.

The prevalence of FXTAS is unknown, although current estimates suggest that about 30%-40% of male FMR1 premutation carriers over 50 years of age, within families already known to have someone with Fragile X, will ultimately exhibit some features of FXTAS. There is no FDA approved therapy for FXTAS and currently used treatments only address the symptoms of the condition, rather than targeting the pathophysiology itself.

Antipurinergic agents constitute a family of compounds that act on purinergic receptors. These receptors are the most abundant receptors in living organisms and appeared early in evolution and are involved in regulating cellular functions. Purinergic receptors are specific classes of membrane receptors that mediate various physiological functions such as the relaxation of certain types of smooth muscle, as a response to the release of adenosine triphosphate (ATP) or adenosine. There are three known distinct classes of purinergic receptors, known as P1, P2X, and P2Y receptors. Also, purinergic signaling is a form of extracellular signaling. This signaling is mediated by purine nucleotides and nucleosides such as adenosine and ATP. This signaling involves the activation of purinergic receptors in the cell and/or in nearby cells, thereby regulating cellular functions.

Chemical compounds that have an effect on purinergic receptors are known. One of these is the compound, suramin, which was first synthesized in the early 1900s. Suramin is a medication used to treat the parasitic disease trypanosomiasis, which is caused by protozoa of the species Trypanosoma brucei and which is more commonly known as African sleeping sickness. The drug is also used to treat river blindness. Because suramin is not orally bioavailable, it is administered by injection into a vein. However, at doses required for treatment of African sleeping sickness, suramin causes a number of side effects. These side effects include nausea, vomiting, diarrhea, abdominal pain, and a feeling of general discomfort. Other side effects include skin sensations such as crawling or tingling sensations, tenderness of the palms and soles, numbness of the extremities, watery eyes, and photophobia. In addition, nephrotoxicity is common, as is peripheral neuropathy when the drug is administered at high doses. Regarding pharmacokinetics, suramin is approximately 99-98% protein bound in the serum and has a half-life of 41-78 days, with an average of 50 days. Also, suramin is not extensively metabolized and is eliminated by the kidneys. Therefore, for suramin to be more effectively used as a treat for a condition such as autism spectrum disorder, FXS, or FXTAS, it would be desirable to minimize the systemic levels of suramin with a targeted delivery to brain tissue.

More recently, it has been reported that suramin exhibits an effect on several multisystem abnormalities in a mouse model of autism spectrum disorder. Also, a small human study was conducted in young boys diagnosed with autism spectrum disorder. See, Antipurinergic Therapy Corrects the Autism-Like Features in the Poly(IC) Mouse Model Robert K. Naviaux, PLoS One. 2013; 8(3): e57380, Published online 2013 Mar. 13. doi: 10.1371/journal.pone.0057380, PMCID: PMC3596371, PMID: 23516405. Also, see, PCT Patent Application Publication No. WO 2018/148580 A1, to Vaughn et al., published Aug. 16, 2018. See, also, Naviaux, R. K. et al., “Low-dose suramin in autism spectrum disorder: a small, phase I/II, randomized clinical trial”, Annals of Clinical and Translational Neurology, 2017 May 26:4(7):491-505.

From the foregoing it is apparent that the treatment of autism spectrum disorders remains challenging. Despite promising results from early animal and human studies, it is recognized that much research is still needed to provide safe and effect delivery of antipurinergic agents, such as suramin, for treating autism. It is necessary to deliver appropriate levels of the drug to brain tissue while also minimizing blood and other tissue levels. However, it is difficult to deliver drugs across the blood-brain barrier (“BBB”), which is a natural protective mechanism of most mammals, including humans. The blood-brain barrier is a highly selective semipermeable border of endothelial cells that prevents solutes in the circulating blood from non-selectively crossing into the extracellular fluid of the central nervous system where neurons reside. Such delivery across the blood-brain barrier is even more challenging for higher molecular weight compounds. Suramin has a molecular weight of approximately 1300 g/mol. A route to attempt to maximize delivery across the blood-brain barrier is to use intranasal delivery to provide higher levels of a drug at the nasal mucosa, with the intent of getting the drug into the blood stream in close proximity to the brain.

It has surprisingly been found in the present invention that the antipurinergic agent, suramin, can potentially be safely and effectively administered intranasally to achieve appropriate level of the drug in brain tissue when certain penetration enhancers are employed. Specifically, it has surprisingly been found that penetration enhancers such as methyl Beta-cyclodextrin, caprylocaproyl macrogol-8 glycerides, and 2-(2-ethoxyethoxy)ethanol are particularly useful for preparing an intranasal suramin formulation having improved penetration of mucosal tissue. These compositions also have the further unexpected benefit of targeting brain tissue, while minimizing systemic blood levels of the suramin drug active. These compositions would therefore have utility for treating neurodevelopmental conditions including, but not limited to, autism spectrum disorder, FXS, FXTAS, chronic fatigue syndrome (CFS), and post-traumatic stress syndrome (PTSD).

### SUMMARY OF THE INVENTION

Methods and compositions for the treatment of cognitive, social, or behavioral disabilities and neurodevelopmental disorders such as autism spectrum disorder, FSX, FXTAS, CFS, and PTSD are described. More specifically, the present invention provides compositions for intranasal administration, i.e. delivery via a nasal route, comprising a therapeutically effective amount of an antipurinergic agent, for example suramin, and pharmaceutically acceptable salts, esters, solvates, and prodrugs thereof. Examples of useful compositions comprise a composition for intranasal administration comprising a therapeutically effective amount of suramin or a pharmaceutically acceptable salt, ester, solvate, or prodrug thereof, a pharmaceutically acceptable carrier, and a penetration aid for delivering therapeutically effective levels of the suramin active to the brain for treating an autism spectrum disorder. These compositions are believed to minimize systemic levels of suramin while targeting brain tissue thereby helping to minimize potential drug toxicity and undesired side effects.

The present invention is based on the surprising discovery that the transmucosal penetration of suramin, as determined in an in vitro assay, was significantly higher when delivered from a formulation comprising various penetration enhancers such as methyl Beta-cyclodextrin, caprylocaproyl macrogol-8 glycerides, and 2-(2-ethoxyethoxy)ethanol. The compositions of the present invention, when administered to mice, were found effective for delivering suramin to brain tissue and demonstrated brain tissue to plasma partitioning ratios. These compositions are designed to deliver the suramin active across the blood-brain barrier to brain tissue, while minimizing systemic levels to less than about a 3 micromolar plasma level and less than about 0.5 micromolar.

The methods of the invention can be achieved through a method that comprises intranasal administration of a single dose of the antipurinergic agent. Alternatively, multiple doses can be administered according to various treatment regimens.

Also provided in the present invention is a device for patient administration or self-administration of the antipurinergic agent comprising a nasal spray inhaler containing an aerosol spray composition of the antipurinergic agent. This composition can comprise the antipurinergic agent and a pharmaceutically acceptable dispersant or solvent system, wherein the device is designed (or alternatively metered) to disperse an amount of the aerosol formulation by forming a spray that contains the dose of the antipurinergic agent. In other embodiments, the inhaler can comprise the antipurinergic agent as a fine powder, and further in combination with particulate dispersants and diluents, or alternatively with the antipurinergic agent combined to be incorporated within particles of the dispersant or to coat the particulate dispersants.

The present invention provides a method for treating cognitive, social, or behavioral disabilities, comprising intranasally delivering a therapeutically effective amount of a pharmaceutical composition comprising a therapeutically effective amount of an antipurinergic agent, or a pharmaceutically acceptable salt, ester, solvate, or prodrug thereof to a patient in need thereof.

In another aspect the present invention provides methods wherein the patient is a human.

In another aspect the present invention provides methods wherein the cognitive, social, or behavioral disability or neurodevelopmental disorder is selected from autism spectrum disorder, FSX, FXTAS, CFS, and PTSD.

In another aspect the present invention provides methods wherein the cognitive, social, or behavioral disability or neurodevelopmental disorder is autism spectrum disorder.

In another aspect the present invention provides methods wherein the cognitive, social, or behavioral disability or neurodevelopmental disorder is FSX.

In another aspect the present invention provides methods wherein the cognitive, social, or behavioral disability or neurodevelopmental disorder is FXTAS.

In another aspect the present invention provides methods wherein the cognitive, social, or behavioral disability or neurodevelopmental disorder is CFS.

In another aspect the present invention provides methods wherein the cognitive, social, or behavioral disability or neurodevelopmental disorder is PTSD.

In another aspect, the present invention provides a method wherein said antipurinergic agent is suramin, or a pharmaceutically acceptable salt, ester, solvate, or prodrug thereof.

In another aspect, the present invention provides a method wherein the pharmaceutically acceptable salt is selected from an alkali metal salt, an alkaline earth metal salt, and an ammonium salt.

In another aspect, the present invention provides a method wherein said salt is a sodium salt.

In another aspect, the present invention provides a method wherein said salt is the hexa-sodium salt.

In another aspect, the present invention provides a method wherein said composition is an aqueous composition.

In another aspect, the present invention provides a method wherein said composition further comprises a penetration enhancer.

In another aspect, the present invention provides a method wherein said penetration enhancer is selected from the group consisting of methyl Beta-cyclodextrin, caprylocaproyl macrogol-8 glycerides, 2-(2-ethoxyethoxy)ethanol, and combinations thereof.

In another aspect, the present invention provides a method wherein said penetration enhancer is methyl beta-cyclodextrin.

In another aspect, the present invention provides a method wherein said penetration enhancer is caprylocaproyl macrogol-8 glycerides.

In another aspect, the present invention provides a method wherein said penetration enhancer is 2-(2-ethoxyethoxy)ethanol.

In another aspect, the present invention provides a method wherein said composition is administered at least once daily.

In another aspect, the present invention provides a method wherein said composition is delivered, i.e. dosed, at least twice daily.

In another aspect, the present invention provides a method wherein said composition is delivered, i.e. dosed, at least twice weekly.

In another aspect, the present invention provides a method wherein said composition is delivered, i.e. dosed, at least once weekly.

In another aspect, the present invention provides a method wherein said composition is delivered, i.e. dosed, at least once biweekly.

In another aspect, the present invention provides a method wherein said composition is delivered, i.e. dosed, at least once monthly, or at least once every 4 weeks.

In another aspect, the present invention provides a method wherein said composition is delivered, i.e. dosed, at least once about every 41 to about 78 days.

In another aspect, the present invention provides a method wherein said composition is delivered, i.e. dosed, at least once about every 50 days.

In another aspect, the present invention provides a method wherein said composition is delivered, i.e. dosed, at least once per a time interval based on the average half-life of suramin.

In another aspect, the present invention provides methods and compositions wherein the amount of suramin is based on the suramin active ingredient (i.e. the chemical entity), using a molecular weight (i.e. a molar mass) of 1297.26 grams/mole.

In another aspect, the present invention provides a method wherein the plasma level of the suramin in the patient is maintained at less than about 3 micromolar (μM), based on the suramin active.

In another aspect, the present invention provides a method wherein the plasma level of the suramin is maintained at less than about 2.75 micromolar, based on the suramin active.

In another aspect, the present invention provides a method wherein the plasma level of the suramin is maintained at less than about 2.5 micromolar, based on the suramin active.

In another aspect, the present invention provides a method wherein the plasma level of the suramin is maintained at less than about 2 micromolar, based on the suramin active.

In another aspect, the present invention provides a method wherein the plasma level of the suramin is maintained at less than about 1 micromolar, based on the suramin active.

In another aspect, the present invention provides a method wherein the plasma level of the suramin is maintained at less than about 0.5 micromolar, based on the suramin active.

In another aspect, the present invention provides a method wherein the brain tissue level of the suramin is from about 1 ng/ml to about 1000 ng/ml.

In another aspect, the present invention provides a method wherein the brain tissue level of the suramin is at least about 1 ng/ml.

In another aspect, the present invention provides a method wherein the brain tissue level of the suramin is at least about 10 ng/ml.

In another aspect, the present invention provides a method wherein the brain tissue level of the suramin is at least about 50 ng/ml.

In another aspect, the present invention provides a method wherein the brain tissue level of the suramin is at least about 100 ng/ml.

In another aspect, the present invention provides a method wherein the brain tissue level of the suramin is at least about 250 ng/ml.

In another aspect, the present invention provides a method wherein the brain tissue level of the suramin is at least about 500 ng/ml.

In another aspect, the present invention provides a method wherein the brain tissue to blood plasma partitioning ratio is at least about 0.05

In another aspect, the present invention provides a method wherein the brain tissue to blood plasma partitioning ratio is at least about 0.1.

In another aspect, the present invention provides a method wherein the brain tissue to blood plasma partitioning ratio is at least about 0.25.

In another aspect, the present invention provides a method wherein the brain tissue to blood plasma partitioning ratio is at least about 0.50.

In another aspect, the present invention provides a method wherein the composition comprises from about 0.01 mg to about 200 mg per unit dosage of suramin, based on the suramin active.

In another aspect, the present invention provides a method wherein the composition comprises from about 0.01 mg to about 100 mg per unit dosage of suramin, based on the suramin active.

In another aspect, the present invention provides a method wherein the composition comprises from about 0.01 mg to about 50 mg per unit dosage of suramin, based on the suramin active.

In another aspect, the present invention provides a method wherein the composition comprises from about 0.01 mg to about 25 mg per unit dosage of suramin, based on the suramin active.

In another aspect, the present invention provides a method wherein the composition comprises from about 0.01 mg to about 10 mg per unit dosage of suramin, based on the suramin active.

In another aspect, the present invention provides a method wherein the composition comprises from about 0.1 mg/kg per week to about 20 mg/kg per week of suramin, based on the suramin active and the weight of the patient.

In another aspect, the present invention provides a method wherein the composition comprises from about 0.025 mg/kg to about 10 mg/kg per unit dosage of suramin, based on the suramin active and the weight of the patient.

In another aspect, the present invention provides a method wherein the composition comprises from about 0.05 mg/kg to about 6 mg/kg per unit dosage of suramin, based on the suramin active and the weight of the patient.

In another aspect, the present invention provides a method wherein the composition comprises from about 0.0476 mg/kg to about 5.720 mg/kg of the per unit dosage of suramin, based on the suramin active and the weight (mass) of the patient.

In another aspect, the present invention provides a method wherein the composition comprises less than about 1 mg/kg per unit dosage of suramin, based on the suramin active and the weight of the patient.

In another aspect, the present invention provides a method wherein the composition comprises less than about 0.5 mg/kg per unit dosage of suramin, based on the suramin active and the weight of the patient.

In another aspect, the present invention provides a method wherein the composition comprises less than about 0.25 mg/kg per unit dosage of suramin, based on the suramin active and the weight of the patient.

In another aspect, the present invention provides a method wherein the composition comprises less than about 0.1 mg/kg per unit dosage of suramin, based on the suramin active and the weight of the patient.

In another aspect, the present invention provides a method wherein the composition comprises less than about 400 mg/m² per unit dosage of suramin, based on the suramin active and the body surface area (BSA) of the patient.

In another aspect, the present invention provides a method wherein the composition comprises less than about 200 mg/m² per unit dosage of suramin, based on the suramin active and the body surface area (BSA) of the patient.

In another aspect, the present invention provides a method wherein the composition comprises less than about 100 mg/m² per unit dosage of suramin, based on the suramin active and the body surface area (BSA) of the patient.

In another aspect, the present invention provides a method wherein the composition comprises less than about 50 mg/m² per unit dosage of suramin, based on the suramin active and the body surface area (BSA) of the patient.

In another aspect, the present invention provides a method wherein the composition comprises less than about 25 mg/m² per unit dosage of suramin, based on the suramin active and the body surface area (BSA) of the patient.

In another aspect, the present invention provides a method wherein the composition comprises from about 10 mg/m² to about 300 mg/m² per unit dosage of suramin, based on the suramin active and the body surface area (BSA) of the patient.

In another aspect, the present invention provides a method wherein the AUC for the plasma level for the suramin active for the patient is less than about 80 μg*day/L.

In another aspect, the present invention provides a method wherein the AUC for the plasma level for the suramin active for the patient is less than about 75 μg*day/L.

In another aspect, the present invention provides a method wherein the AUC for the plasma level for the suramin active for the patient is less than about 50 μg*day/L.

In another aspect, the present invention provides a method wherein the AUC for the plasma level for the suramin active for the patient is less than about 25 μg*day/L.

In another aspect, the present invention provides a method wherein the AUC for the plasma level for the suramin active for the patient is less than about 10 μg*day/L.

In another aspect, the present invention provides a method wherein the Cₘₐₓ for the plasma level for the su amin active for the patient is less than about 75 micromolar, per dose of drug composition.

In another aspect, the present invention provides a method wherein the Cₘₐₓ for the plasma level for the suramin active for the patient is less than about 7.5 micromolar, per dose of drug composition.

In another aspect, the present invention provides a method wherein the Cₘₐₓ for the plasma level for the suramin active for the patient is less than about 0.1 micromolar. Although there is no minimum Cₘₐₓ the amount can generally be above about 0.01 micromolar per dose of drug composition.

In another aspect, the present invention provides a method wherein said composition is in the form of a nasal spray, i.e. a spray for intranasal administration.

In another aspect, the present invention provides a method wherein each unit dosage comprises about 0.01 ml to about 0.5 ml of liquid.

In another aspect, the present invention provides a method wherein each unit dosage comprises about 0.1 ml of liquid.

In another aspect, the present invention provides a method wherein the composition exhibits, i.e. is capable of providing, a penetration rate of about 1 micrograms/cm² per hour to about 200 micrograms/cm² per hour of suramin, based on the suramin active, through cultured human airway tissue.

In another aspect, the present invention provides a method wherein the composition further comprises an agent selected for osmolality control.

In another aspect, the present invention provides a method wherein the composition further comprises an agent selected for osmolality control, wherein said agent is selected from a salt, such as for example sodium chloride.

In another aspect, the present invention provides a method wherein the compositions further comprises a thickening agent.

In another aspect, the present invention provides a method wherein said autism spectrum disorder is selected from the group consisting of autistic disorder, childhood disintegrative disorder, pervasive developmental disorder-not otherwise specified (PDD-NOS), and Asperger syndrome.

In another aspect, the present invention provides a method wherein said autism spectrum disorder includes one or more symptoms selected from difficulty communicating, difficulty interacting with others, and repetitive behaviors.

In another aspect, the present invention provides a method wherein treating said autism spectrum disorder, FXS, FXTAS, CFS or PTSD comprises improving more or more symptoms relative to symptoms of said patient prior to said administration, wherein said one or more symptoms are selected from difficulty communicating, difficulty interacting with others, and repetitive behaviors.

In another aspect, the present invention provides a method wherein treating said autism spectrum disorder, FXS, FXTAS, CFS or PTSD comprises improving an assessment score of said patient relative to a score from said patient prior to said administration.

In another aspect, the present invention provides a method wherein an assessment score of said patient is improved by 10% or more relative to a score from said patient prior to said administration.

In another aspect, the present invention provides a method wherein the assessment score is selected from ABC, ADOS, ATEC, CARS CGI, and SRS.

In another aspect, the present invention provides a method wherein an ADOS score of the patient is improved by 1.6 or more relative to a score prior to said administration, or a corresponding performance improvement on a similar test.

In another aspect, the present invention provides a method wherein the p-value of improvement of said ADOS score or similar test is 0.05 or less.

In another aspect, the present invention provides a method wherein the size effect of improvement of said ADOS score or similar test is about 1 or more.

In another aspect, the present invention provides a method wherein the size effect of improvement of said ADOS score or similar test is about 2.9 or more.

In another aspect, the present invention provides a method for treating an autism spectrum disorder, FXS, FXTAS, CFS or PTSD comprising administering a therapeutically effective amount of a pharmaceutical composition comprising a therapeutically effective amount of an antipurinergic agent, or a pharmaceutically acceptable salt, ester, solvate, or prodrug thereof to a human in need thereof, wherein the plasma level of the antipurinergic agent is maintained at less than about 3 micromolar, or less than about 1 micromolar, or less than about 0.5 micromolar.

In another aspect, the present invention provides an intranasal delivery pharmaceutical composition for treating an autism spectrum disorder, FXS, FXTAS, CFS or PTSD comprising:

(a) therapeutically effective amount of an antipurinergic agent, or a pharmaceutically acceptable salt, ester, solvate, or prodrug thereof, and

(b) a penetration enhancer.

In another aspect, the present invention provides a composition further comprising (c) water.

In another aspect, the present invention provides a composition wherein the antipurinergic agent is suramin, or a pharmaceutically acceptable salt, ester, solvate, or prodrug thereof.

In another aspect, the present invention provides a composition such that when the composition is administered to a human in need thereof the plasma level of the suramin is maintained at less than about 3 micromolar, based on the suramin active.

In another aspect, the present invention provides a composition such that when the composition is administered to a human in need thereof the plasma level of the suramin is maintained at less than about 1 micromolar, or less than about 0.5 micromolar, based on the suramin active.

In another aspect, the present invention provides the use of suramin, or a pharmaceutically acceptable salt, ester, solvate, or prodrug thereof in the manufacture of a medicament for intranasal delivery of a therapeutically effective amount of suramin for treating an autism spectrum disorder, FXS, FXTAS, CFS or PTSD in a patient, e.g., a human, in need thereof.

In another aspect, the present invention provides a use such that the plasma level of the suramin is maintained at less than about 3 micromolar, or less than about 1 micromolar, or less than about 0.5 micromolar, based on the suramin active.

In another aspect, the present invention provides a device for patient administration, including administration selected from self-administration and administration to the patient by an individual other than the patient, comprising a nasal spray inhaler for administering a composition comprising an antipurinergic agent, wherein the device is designed (or alternatively metered) to disperse an amount of the antipurinergic agent for treating an autism spectrum disorder, FXS, FXTAS, CFS or PTSD in a patient in need thereof.

In another aspect the, the present invention provide a device wherein the antipurinergic agent comprises a composition selected from a solution, an emulsion, or a powder.

These and other aspects of the present invention will become apparent from the disclosure herein.

### BRIEF DESCRIPTION OF THE DRAWINGS

FIG. 1 shows a plot of cumulative drug penetration, in mg, versus time, in hours, for aqueous suramin compositions with three different penetration enhancers versus a control composition with no penetration enhancer.

FIG. 2 shows a plot of cumulative drug penetration, in mg, versus time, in hours, for aqueous suramin compositions with five different penetration enhancers versus a control composition with no penetration enhancer.

FIG. 3 shows a plot of the total concentration, in ng/ml, of suramin in plasma versus brain tissue in mice when administered by intraperitoneal (IP) injection, 20 mg/kg, weekly to the mice beginning at 9 weeks of age and continuing for four weeks (i.e. given at age weeks 9, 10, 11 and 12).

FIG. 4 shows a plot comparing the total concentration, in ng/ml, of suramin in plasma versus brain tissue in mice when administered intranasally (IN) daily for 28 days. A composition of the present invention comprising IN suramin, at a concentration of 100 mg/mL×6 mL per spray, was administered as one spray per nostril, one time per day, (interval of each application is around 2 minutes to ensure absorption) for 28 days (total of 56 sprays over 28 day period) beginning at 9 weeks of age (i.e. given daily during age weeks 9, 10, 11 and 12).

FIG. 5 shows a plot comparing the total concentration, in ng/ml, of suramin in plasma versus brain tissue in mice when administered intranasally (IN) every other day for 28 days. A composition of the present invention comprising IN suramin, at a concentration of 100 mg/mL×6 mL per spray, was administered as one spray per nostril, every other day, (interval of each application is around 2 minutes to ensure absorption) for 28 days (total of 28 sprays over 28 day period) beginning at 9 weeks of age (i.e. given daily during age weeks 9, 10, 11 and 12).

FIG. 6 shows a plot comparing the total concentration, in ng/ml, of suramin in plasma versus brain tissue in mice when administered intranasally (IN) once per week for 4 weeks.

A composition of the present invention comprising IN suramin, at a concentration of 100 mg/mL×6 mL per spray, was administered as one spray per nostril, one time per week, (interval of each application is around 2 minutes to ensure absorption) for 4 weeks (28 days) (total of 8 sprays over 28 day period) beginning at 9 weeks of age (i.e. given daily during age weeks 9, 10, 11 and 12).

FIG. 7 shows a plot comparing the total percentage of suramin in plasma in mice when administered by intraperitoneal (IP) injection once weekly for 4 weeks (28 days), intranasally (IN) daily for 28 days, intranasally (IN) every other day for 28 days, and intranasally (IN) once per week for 4 weeks (28 days).

FIG. 8 shows a plot comparing the total percentage of suramin in brain tissue in mice when administered by intraperitoneal (IP) injection once weekly for 4 weeks (28 days), intranasally (IN) daily for 28 days, intranasally (IN) every other day for 28 days, and intranasally (IN) once per week for 4 weeks (28 days).

FIG. 9 shows a plot comparing the total percentage of suramin in plasma versus brain tissue in mice when administered by intraperitoneal (IP) injection once weekly for 4 weeks (28 days), intranasally (IN) daily for 28 days, intranasally (IN) every other day for 28 days, and intranasally (IN) once per week for 4 weeks (28 days).

FIG. 10 shows a plot comparing the brain tissue to plasma partitioning ration of suramin in mice when administered by intraperitoneal (IP) injection once weekly for 4 weeks (28 days), intranasally (IN) daily for 28 days, intranasally (IN) every other day for 28 days, and intranasally (IN) once per week for 4 weeks (28 days).

### DETAILED DESCRIPTION OF THE INVENTION

### Definitions

As used herein, the following terms and abbreviations have the indicated meanings unless expressly stated to the contrary.

The term “ABC”, as used herein is also known as the “Aberrant Behavior Checklist” and is a rating scale for evaluating autism.

The term “ADOS”, as used herein is also known as “The Autism Diagnostic Observation Schedule” is an instrument for diagnosing and assessing autism. The protocol consists of a series of structured and semi-structured tasks that involve social interaction between the examiner and the person under assessment.

The term “ATEC”, as used herein is also known as “The Autism Treatment Evaluation Scale”, is a 77-item diagnostic assessment tool that was developed at the Autism Research Institute. The ATEC was originally designed to evaluate the effectiveness of autism treatments, but is also used as a screening tool.

The term “AUC”, also known as “Area Under the Curve” as used herein is standard terminology in pharmacology, specifically pharmacokinetics. The term refers to the definite integral of a curve that describes the variation of a drug concentration in blood plasma as a function of time. In practice, the drug concentration is measured at certain discrete points in time and the trapezoidal rule is used to estimate AUC. The AUC gives a measure of bioavailability and refers to the fraction of drug absorbed systemically. Knowing this, one can also determine the clearance for the drug. The AUC reflects the actual body exposure to drug after administration of a dose of the drug and is usually expressed in mg*h/L or μg*h/L (where “h” stands for hours). Alternatively, the AUC can be expressed in mg*day/L or μg*day/L.

The term “based on the suramin active” as used herein is meant to provide a basis for determining or calculating the amount of suramin based on the suramin molecular weight (i.e. a molar mass) of 1297.26 grams/mole. This is an important consideration for determining the amount of suramin when it is delivered as a salt or other form, having a different total molecular weight, such as for example the hexa-sodium salt which would have a molecular weight (i.e. a molar mass) of 1429.15 grams/mole.

The term “CARS”, as used herein is also known as “The Childhood Autism Rating Scale” and is a behavior rating scale intended to help diagnose and evaluate autism.

The term “CFS”, as used herein is also know as “Chronic Fatigue Syndrome”.

The term “CGI”, as used herein is also known as “The Clinical Global Impression” rating scale and is a measure of symptom severity, treatment response and the efficacy of treatments in treatment studies of patients with psychological disorders.

The term “Cₘₐₓ” as used herein is standard terminology in pharmacology, specifically pharmacokinetics, for defining the maximum (or peak) serum concentration that a drug achieves in a specified compartment or test area of the body after the drug has been administered and before the administration of a second dose.

The term “FXS” as used herein means fragile X syndrome.

The term “FXTAS” as used herein means fragile X-associated tremor/ataxia syndrome.

The term “IN” as used herein means intranasal.

The term “pharmaceutically acceptable” is used herein with respect to the compositions, in other words the formulations, of the present invention, and also with respect to the pharmaceutically acceptable salts, esters, solvates, and prodrugs of suramin. The pharmaceutical compositions of the present invention comprise a therapeutically effective amount of suramin and a pharmaceutically acceptable carrier. These carriers can contain a wide range of excipients. Pharmaceutically acceptable carriers are those conventionally known carriers having acceptable safety profiles. The compositions are made using common formulation techniques. See, for example, Remington's Pharmaceutical Sciences, 17th edition, edited by Alfonso R. Gennaro, Mack Publishing Company, Easton, Pa., 17th edition, 1985. Regarding pharmaceutically acceptable salts, these are described below.

The term “PTSD”, as used herein is also known as “Post-Traumatic Stress Syndrome”.

The term “SRS”, as used herein is also known as The “Social Responsiveness Scale” which is used herein is a measure of autism spectrum disorder.

The term “subject” means a human patient or animal in need of treatment or intervention for an autism spectrum disorder.

The term “therapeutically effective” means an amount of suramin needed to provide a meaningful or demonstrable benefit, as understood by medical practitioners, to a subject, such as a human patient in need of treatment. Conditions, intended to be treated include, for example, autistic disorder, childhood disintegrative disorder, pervasive developmental disorder-not otherwise specified (PDD-NOS), and Asperger syndrome. For example, a meaningful or demonstrable benefit can be assessed or quantified using various clinical parameters. The demonstration of a benefit can also include those provided by models, including but not limited to in vitro models, in vivo models, and animal models. An example of such an in vitro model is the permeation of the drug active studied using cultured human airway tissues (EpiAirway AIR-100) to simulate permeation across the nasal mucosal membrane.

The term “intranasal” (“IN”) as used herein with respect to the pharmaceutical compositions and actives therein, means a composition that is administered through the nose for delivery across the mucosal membrane inside the nasal cavity. This membrane is a well vascularized thin mucosa. Furthermore, this mucosa is in close proximity to the brain and provides a means to maximize the transport of drugs across the blood-brain barrier. The blood-brain barrier is a highly selective semipermeable border that separates the circulating blood from the brain and extracellular fluid in the central nervous system. Delivering therapeutic agents to specific regions of the brain presents a challenge to treatment of many brain disorders. It should be noted that transmucosal administration is different from topical administration and transdermal administration. The U.S. Food & Drug Administration has provided a standard for a wide range of routes of administration for drugs, i.e. “Route of Administration”. The following definitions are provided by the FDA for example for endosinusial, intracerebral, intranasal, nasal, topical, transdermal, and transmucosal routes of drug administration. The routes of administration useful in the present invention include endosinusial, intranasal, and nasal, recognizing that transmucosal delivery through the nasal mucosa is also intended. These routes of administration are distinguished from inhalation which is intended to deliver a drug into the lungs and bronchi. See for example, U.S. Pat. No. 8,785,500 to Charney et al., issued Jul. 22, 2014, which discloses examples of methods and compositions for intranasally administering a drug active.

| | | | | NCI* |
|---|---|---|---|---|
| | | SHORT | FDA | CONCEPT |
| NAME | DEFINITION | NAME | CODE | ID |
| ENDO- | Administration within | E-SINUS | 133 | C38206 |
| SINUSIAL | the nasal sinuses of | | | |
| | the head. | | | |
| INTRA- | Administration within | I-CERE | 404 | C38232 |
| CEREBRAL | the cerebrum. | | | |
| INTRA- | Administration within | I-SINAL | 010 | C38262 |
| SINAL | the nasal or periorbital | | | |
| | sinuses. | | | |
| NASAL | Administration to the | NASAL | 014 | C38284 |
| | nose; administered by | | | |
| | way of the nose. | | | |
| TOPICAL | Administration to a | TOPIC | 011 | C38304 |
| | particular spot on the | | | |
| | outer surface of | | | |
| | the body. | | | |
| TRANS- | Administration | T-DERMAL | 358 | C38305 |
| DERMAL | through the | | | |
| | dermal layer of the | | | |
| | skin to the systemic | | | |
| | circulation by | | | |
| | diffusion. | | | |
| TRANS- | Administration across | T-MUCOS | 122 | C38283 |
| MUCOSAL | the mucosa. | | | |

*National Cancer Institute

See, https://www.fda.gov/Drugs/DevelopmentApprovalProcess/FormsSubmissionRequirements/ElectronicSubmissions/DataStandardsManualmonographs/um071667.htm

The terms “treat,” “treating” or “treatment,” as used herein, include alleviating, abating or ameliorating the condition, e.g. autism and other central nervous system disorders, or preventing or reducing the risk of contracting the condition or exhibiting the symptoms of the condition, ameliorating or preventing the underlying causes of the symptoms, inhibiting the condition, arresting the development of the condition, relieving the condition, causing regression of the condition, or stopping the symptoms of the condition, either prophylactically and/or therapeutically.

The methods of treatment using suramin or a pharmaceutically acceptable salt, ester, solvate, or prodrug thereof or the pharmaceutical compositions of the present invention, in various embodiments also include the use of suramin or a pharmaceutically acceptable salt, ester, solvate, or prodrug thereof in the manufacture of a medicament for the desired treatment, such as for an autism spectrum disorder.

Suramin

The present invention utilizes a therapeutically effective amount of the antipurinergic agent suramin, or a pharmaceutically acceptable salt, ester, solvate, or prodrug thereof, a penetration enhancer, and also a pharmaceutically acceptable carrier for providing intranasal administration for treating an autism spectrum disorder.

Suramin is a sulfonic acid drug compound, corresponding to the CAS Registry Number 145-63-1 and ChemSpider ID 5168. One of the chemical names for suram in is: 1,3,5-Naphthalenetrisulfonic acid, 8,8′-[carbonylbis[imino-3,1-phenylenecarbonylimino(4-methyl-3,1-phenylene)carbonylimino]bis-. The compound is a medication used to treat African sleeping sickness and river blindness and is known by the trade names Antrypol, 309 F, 309 Fourneau, Bayer 205, Germanin, Moranyl, Naganin, and Naganine. However, the drug is not approved by the US FDA. The drug is administered by venous injection. Suramin has been reported to have been studied in a mouse model of autism and in a Phase I/II human trial. See, Naviaux, J. C. et al., “Reversal of autism-like behaviors and metabolism in adult mice with single-dose antipurinergic therapy”. Translational Psychiatry. 4 (6): e400 (2014). Also, see, Naviaux, R. K. et al., “Low-dose suramin in autism spectrum disorder: a small, phase I/II, randomized clinical trial”, Annals of Clinical and Translational Neurology, 2017 May 26:4(7):491-505.

Suramin is reported to have a half-life of between about 41 to 78 days with an average of 50 days. See, Phillips, Margaret A.; Stanley, Jr, Samuel L. (2011). “Chapter 50: Chemotherapy of Protozoal Infections: Amebiasis, Giardiasis, Trichomoniasis, Trypanosomiasis, Leishmaniasis, and Other Protozoal Infections”. In Brunton, Laurence L. Chabner, Bruce A.; Knollmann, Bjorn Christian (eds.). Goodman and Gilman's The Pharmacological Basis of Therapeutics (12th ed.). McGraw Hill. pp. 1437-1438.

The chemical formula of suramin is C₅₁H₄₀N₆O₂₃S₆. Suramin therefore has a molecular weight (i.e. a molar mass) of 1297.26 grams/mole. Suramin is usually delivered as a sodium sulfonate salt, such as the hexa-sodium salt, which has a molecular weight (i.e. a molar mass) of 1429.15 grams/mole. Note that these molecular weight values will vary slightly depending on what atomic weight values are used for the calculations. The chemical structure for suramin is shown immediately below.

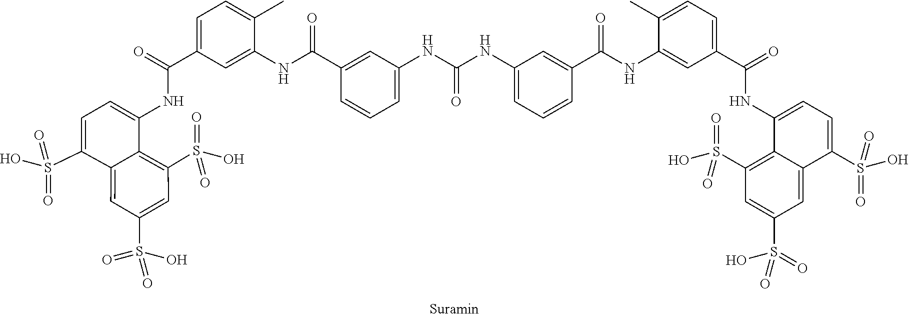 [SMILES: Cc1ccc(C(=O)Nc2ccc(S(=O)(=O)O)c3cc(S(=O)(=O)O)cc(S(=O)(=O)O)c23)cc1NC(=O)c1cccc(NC(=O)Nc2cccc(C(=O)Nc3cc(C(=O)Nc4ccc(S(=O)(=O)O)c5cc(S(=O)(=O)O)cc(S(=O)(=O)O)c45)ccc3C)c2)c1]

Pharmaceutically acceptable salts, esters, solvates, and prodrugs of suramin are useful for the methods and compositions of the present invention. As used herein, “pharmaceutically acceptable salts, esters, solvates, and prodrugs” refer to derivatives of suramin. Examples of pharmaceutically acceptable salts include, but are not limited to, alkali metal salts, alkaline earth metal salts, and ammonium salts. Examples of alkali metal salts include lithium, sodium, and potassium salts. Examples of alkaline earth metal salts include calcium and magnesium salts. The ammonium salt, NH4⁺. itself can be prepared, as well as various monoalkyl, dialkyl, trialkyl, and tetraalkyl ammonium salts. Also, one or more of the alkyl groups of such ammonium salts can be further substituted with groups such as hydroxy groups, to provide an ammonium salt of an alkanol amine. Ammonium salts derived from diamines such as 1,2-diaminoethane are contemplated herein. The hexa-sodium salt of suramin is useful herein.

The pharmaceutically acceptable salts, esters, solvates, and prodrugs of suramin can be prepared from the parent compound by conventional chemical methods. Generally, the salts can be prepared by reacting the free acid form of the compound with a stoichiometric amount of the appropriate base in water or in an organic solvent, or in a mixture of the two; generally, non-aqueous media like ether, ethyl acetate, ethanol, isopropanol, or acetonitrile are preferred. The esters of suramin can be prepared by reacting the parent compound with an alcohol, and removal of water formed from the reaction. Alternatively, other methods can be used. Anywhere from one up to all six of the sulfonate groups of suramin can be esterified to form a mono-ester up to a hexa-ester. Examples of these esters include the mesylate (methanesulfonate), CH₃SO₃—; triflate (trifluoromethanesulfonate), CF₃SO₃—; ethanesulfonate (esilate, esylate), C₂H₅SO₃—; tosylate (p-toluenesulfonate), CH₃C₆H₄SO₃—; benzenesulfonic acid (besylate), C₆H₅SO₃—; closilate (closylate, chlorobenzenesulfonate), ClC₆H₄SO₃—;camphorsulfonate (camsilate, camsylate), (C₁₀H₁₅O)SO₃—; pipsylate (p-iodobenzenesulfonate derivative); and nosylate (p-nitrobenzensulfonate derivate).

The solvates of suramin means that one or more solvent molecules are associated with one or more molecules of suramin, including fraction solvates such as, e.g., 0.5 and 2.5 solvates. The solvents can be selected from a wide range of solvents including water, ethanol, isopropanol, and the like. The prodrugs of suramin can be prepared using convention chemical methods, depending on the prodrug chosen. A prodrug is a medication or compound that, after administration, is metabolized (i.e., converted within the body) into a pharmacologically active drug. Prodrugs can be designed to improve bioavailability when a drug itself is poorly absorbed from the gastrointestinal tract. Prodrugs are intended to include covalently bonded carriers that release an active parent drug of the present invention in vivo when such prodrug is administered. In some classifications, esters are viewed as prodrugs, such as the esters of suramin described herein. Other types of prodrugs can include sulfonamide derivatives and anhydrides.

Furthermore, the various esters and prodrugs can include further derivatization to make polyethylene glycol (PEG) and polypropylene glycol (PPG) derivatives and mixed derivatives, an example of which would a pegylated derivative.

Dosages

For treating African sleeping sickness, suramin is typically administered according to a treatment regimen with five intravenous injections of 20 mg/kg of the drug, every 3-7 days over a total period of 4 weeks. Note that this dosage of suramin for treating African sleeping sickness is relatively high and that the treatment regimen requires relatively frequent dosing, both of which have the potential for causing drug toxicity and adverse reactions. The potential for such toxicity and adverse reactions would be less tolerated for treating a condition such as autism spectrum disorder, FXS, or FXTAS, particularly in children, compared to the acute and potentially life-threatening African sleeping sickness.

For the present invention for treating autism spectrum disorder, dosages of suramin in the compositions administered will be in the range of about 0.01 mg to about 200 mg per dose, or about 0.01 mg to about 100 mg per dose, such as a dose of a nasal spray, based on the suramin active, where each administered spray dose would comprise about 0.1 ml of liquid.

Compositions can also be determined on a weight basis. In one embodiment the compositions useful here comprise from about 0.01% to about 60% by weight suramin or a pharmaceutically salt, ester, solvate or, prodrug thereof, based on the weight of the suramin active. In another embodiment these compositions here comprise from about 0.1% to about 25% by weight suramin or a pharmaceutically salt, ester, solvate or, prodrug thereof, based on the weight of the suramin active

For these foregoing compositions comprising a designated amount or weight percentage of the suramin or the amount or weight percentage of the suramin is determined or calculated based on the actual amount of the suramin moiety, which has a molar mass of 1297.26 grams/mole, and not including the additional weight provided by any counter ions, or ester, solvate or prodrug moieties when a suramin salt, ester, solvate, or prodrug is used. In other words, the compositions are based on the amount or weight percentage of the suramin chemical moiety.

Furthermore, because the present invention is related to intranasal delivery compositions and because it is highly desirable to limit systemic exposure, the unit dosage could be formulated to limit the systemic plasma levels of the suramin. Generally, it would be desirable to maintain the suramin plasma levels below a concentration of about 3 micromolar. In further embodiments it would be desirable to maintain the suramin plasma levels below a concentration of about 2 micromolar. In further embodiments it would be desirable to maintain the suramin plasma levels below a concentration of about 1 micromolar. In further embodiments it would be desirable to maintain the suramin plasma levels below a concentration of about 0.1 micromolar. In further embodiments it would be desirable to maintain the suramin plasma levels below a concentration of about 0.05 micromolar. In further embodiments it would be desirable to maintain the suramin plasma levels below a concentration of about 0.01 micromolar. Although a minimum systemic suramin plasma level may not be necessary as long as the appropriate brain blood and tissue levels are maintained, it may generally be desirable that the suramin plasma levels be greater than about 1 nanomolar.

Furthermore, because the present invention is related to intranasal compositions and methods of treatment it is highly desirable to limit systemic exposure of the suramin to minimize the potential for drug toxicity and undesired side effects and to maintain an appropriate window of safety. This limitation of systemic levels can be achieved by controlling the PK/PD profile. In some embodiments, the unit dosage should demonstrate at least one of the following blood plasma pharmacokinetic parameters for delivery of that unit dosage: a Cₘₐₓ less than about 75 micromolar (i.e. μM), or less than about 7.5 micromolar, or less than about 0.1 micromolar, or an AUC less than about 80 μg*day/L, or less than about 75 μg*day/L, or less than about 50 μg*day/L, or less than about 25 μg*day/L, or less than about 10 μg*day/L. The Cₘₐₓ can be above at least about 0.01 micromolar. The Cₘₐₓ values can be converted from micromolar to ng/ml (based on the suramin active using a molecular weight of 1297.26 grams/mole) meaning that 1 micromolar is equivalent to 1297.26 ng/ml. Should one want to have the amount based on the hexa-sodium salt a value of 1429.15 grams/mole can be used for the conversion calculation.

Methods of Treatment and Dosing Regimens

The present invention utilizes a therapeutically effective amount of suramin or a pharmaceutically acceptable salt thereof and a pharmaceutically acceptable carrier for treating autism spectrum disorders, FXS, or FXTAS, and other neurological conditions.

The methods comprise nasally administering a therapeutically effective amount of suramin, or a pharmaceutically acceptable salt, ester, solvate, or prodrug thereof to a human patient, in need thereof.

Various dosing regimens can be prescribed and used based on the skill and knowledge of the physician or other practitioner. In some embodiments, a unit dosage of the composition, as described herein can be applied at least once daily. In other embodiments, a unit dosage of the composition can be applied at least twice daily, or at least once weekly, or at least twice weekly. Based on the pharmacokinetic and pharmacodynamic parameters of suramin, the dosing amount and regimen can be appropriately varied. Suramin is approximately 99-98% protein bound in the serum and has a half-life of 41-78 days with an average of 50 days.

Therapy can be continued in the judgment of the physician or practitioner until the desired therapeutic benefit is achieved. In some instances, it can be desirable to continue long term or maintenance therapy.

Evaluation of Treatments

The present invention provides a method wherein said autism spectrum disorder, FXS, FXTAS, CFS or PTSD includes one or more symptoms selected from difficulty communicating, difficulty interacting with others, disruptive and repetitive behaviors. Patients with autism spectrum disorder, FXS, FXTAS, CFS or PTSD can be evaluated using a variety of rating scales to determine the level of severity of their disorder and any improvements or changes upon administration of a treatment.

For example, the present invention provides a method wherein treating the autism spectrum disorder, FXS, FXTAS, CFS or PTSD comprises improving more or more symptoms of the patient relative to the symptoms prior to therapy. The improvement can be determined by comparing an assessment score of the patient's symptoms relative to a score from the patient's symptoms prior to said administration. It is desirable to provide an improvement of 10% or more relative to a score from the patient prior to administration of the treatment.

Examples of assessment scales for evaluating autism spectrum disorder include those selected from ABC, ADOS, ATEC, CARS CGI, and SRS.

The term “ABC” is also known as the “Aberrant Behavior Checklist” and is a rating scale for evaluating autism. The term “ADOS” is also known as “The Autism Diagnostic Observation Schedule”. The protocol consists of a series of structured and semi-structured tasks that involve social interaction between the examiner and the person under assessment. The term “ATEC” is also known as “The Autism Treatment Evaluation Scale”, and is a 77-item diagnostic assessment tool that was developed at the Autism Research Institute. The ATEC was originally designed to evaluate the effectiveness of autism treatments, but is also used as a screening tool. The term “CARS” is also known as “The Childhood Autism Rating Scale” and is a behavior rating scale intended to help diagnose and evaluate autism. The term “CGI” is also known as “The Clinical Global Impression” rating scale and is a measure of symptom severity, treatment response and the efficacy of treatments in treatment studies of patients with psychological disorders. The term “SRS” is also known as The “Social Responsiveness Scale” which is used herein and is a measure of autism spectrum disorder.

For example, the present invention provides a method wherein an ADOS score of the patient is improved by 1.6 or more relative to a score prior to administration of treatment, or a corresponding performance improvement on a similar test. Furthermore, the present invention provides a method wherein the p-value of improvement of ADOS score or similar test is 0.05 or less. In another aspect, the present invention provides a method wherein the size effect of improvement of the ADOS score or similar test is about 1 or more or is up to about 2.9 or more.

Formulations for Intranasal Administration and Penetration Enhancers

The target indication of the invention composition is related to autism, FXS, and FXTAS, and other central nervous system diseases. As such, efforts are made to provide formulations that can readily reach the brain areas by crossing the blood-brain barrier. A feasible route of administration is delivery via the nasal cavity by a nasal drug delivery system, i.e. an intranasal (IN) formulation spray.

Useful compositions for intranasal delivery can be in the form of nasal sprays. These compositions can have the active in the form of aqueous compositions. In other embodiments, the active agent can be a fine powder, and further in combination with particulate dispersants and diluents, or alternatively combined to form or coat the particulate dispersants. These compositions would generally be on the order of about 0.01 ml to about 0.5 ml, with a target volume of about 0.1 ml per spray. One to two sprays could be applied to provide a unit dosage.

The pharmaceutical compositions herein can comprise a penetration enhancer. Surprisingly, the following penetration enhancers have been found to increase the transmucosal tissue penetration of suramin: methyl Beta-cyclodextrin, caprylocaproyl macrogol-8 glycerides, and 2-(2-ethoxyethoxy)ethanol. The material methyl Beta-cyclodextrin (methyl-beta-cyclodextrin) is also known by the CAS Registry Number 128446-36-6 and the trade name methyl betadex. The material caprylocaproyl macrogol-8 glycerides is also known as caprylocaproyl polyoxyl-8 glycerides and PEG-8 caprylic/capric glycerides, by the CAS Registry Number 85536-07-8, and the trade name Labrasol®. The material 2-(2-ethoxyethoxy)ethanol is also known as diethylene glycol ethyl ether, by the CAS Registry Number 111-90-0, and by the trade names Carbitol™ and Transcutol® P.

The penetration enhance is generally used at about 40% by weight of the composition. Other useful ranges are from about 0.1% to about 90% by weight of the composition, or from about 1% to about 80% by weight of the composition, or from about 10% to about 75% by weight of the composition, or from about 25% to about 50% by weight of the composition.

The water in the composition is usually Q.S. The abbreviation QS stands for Quantum satis and means to add as much of the ingredient, in this case water, to achieve the desired result, but not more.

Other ingredients can include various salts for osmolality control and thickening agents.

In some embodiment compositions can comprise the following functional ingredients:

1. Active ingredient: suramin, in concentration of 10 to 200 mg/mL

2. A solvent/carrier, e.g. water

3. A tissue permeation enhancer

4. A preservative(s)

5. A thickener to modify the spray solution viscosity, and

6. A buffering (pH adjusting) or osmolarity agent.

These formulations can be made using standard formulation and mixing techniques familiar to one of ordinary skill in the art of pharmaceuticals and formulations.

In one embodiment, the compositions or formulations of the present invention comprise suramin or a pharmaceutically acceptable salt, ester, solvate, or prodrug thereof and a pharmaceutically acceptable carrier. These formulations can be made using standard formulation and mixing techniques familiar to one of ordinary skill in the art of pharmaceuticals and formulations.

In one aspect, the pharmaceutical composition is selected from a solution, suspension, or dispersion for administration as a spray or aerosol. In other aspects the formulation can be delivered as drops by a nose dropper or applied directly to the nasal cavity. Other pharmaceutical compositions are selected from the group consisting of a gel, ointment, lotion, emulsion, cream, foam, mousse, liquid, paste, jelly, or tape, that is applied to the nasal cavity.

Useful herein are compositions wherein the pharmaceutically acceptable carrier is selected from water or mixtures of water with other water-miscible components. In the case of emulsions, the components do not have to be miscible with water.

In other embodiments the compositions can comprise a buffer to maintain the pH of the drug formulation, a pharmaceutically acceptable thickening agent, humectant and surfactant. Buffers that are suitable for use in the present invention include, for example, hydrochloride, acetate, citrate, carbonate and phosphate buffers.

The viscosity of the compositions of the present invention can be maintained at a desired level using a pharmaceutically acceptable thickening agent. Thickening agents that can be used in accordance with the present invention include for example, xanthan gum, carbomer, polyvinyl alcohol, alginates, acacia, chitosans, sodium carboxyl methylcellulose (Na CMC) and mixtures thereof. The concentration of the thickening agent will depend upon the agent selected and the viscosity desired.

The compositions of the present invention also include a tolerance enhancer to reduce or prevent drying of the mucus membrane (humectants) and to prevent irritation thereof. Suitable tolerance enhancers that can be used in the present invention include, for example, humectants, sorbitol, propylene glycol, mineral oil, vegetable oil and glycerol; soothing agents, membrane conditioners, sweeteners and mixtures thereof. The concentration of the tolerance enhancer(s) in the present compositions will also vary with the agent selected.

In order to enhance absorption of the drug through the nasal mucosa, a therapeutically acceptable surfactant may be added to the intranasal formulation. Suitable surfactants that can be used in accordance with the present invention include, for example, polyoxyethylene derivatives of fatty acid partial esters of sorbitol anhydrides, such as for example, Tween 80, Polyoxyl 40 Stearate, Polyoxy ethylene 50 Stearate, fusidates, bile salts and Octoxynol. Suitable surfactants include non-ionic, anionic and cationic surfactants. These surfactants can be present in the intranasal formulation in a concentration ranging from about 0.001% to about 20% by weight.

In the present invention other optional ingredients may also be incorporated into the nasal delivery system provided they do not interfere with the action of the drug or significantly decrease the absorption of the drug across the nasal mucosa. Such ingredients can include, for example, pharmaceutically acceptable excipients and preservatives. The excipients that can be used in accordance with the present invention include, for example, bio-adhesives and/or swelling/thickening agents.

In the present invention, any other suitable absorption enhancers as known in the art may also be used.

Preservatives can also be added to the present compositions. Suitable preservatives that can be used with the present compositions include, for example, benzyl alcohol, parabens, thimerosal, chlorobutanol and benzalkonium, with benzalkonium chloride being preferred. Typically, the preservative will be present in the present compositions in a concentration of up to about 2% by weight. The exact concentration of the preservative, however, will vary depending upon the intended use and can be easily ascertained by one skilled in the art.

The absorption enhancing agent includes (i) a surfactant; (ii) a bile salt (including sodium taurocholate); (iii) a phospholipid additive, mixed micelle, or liposome; (iv) an alcohol (including a polyol as discussed above, for example, propylene glycol or polyethylene glycol such as PEG 3000, etc.); (v) an enamine; (vi) a nitric oxide donor compound; (vii) a long-chain amphipathic molecule; (viii) a small hydrophobic uptake enhancer; (ix) sodium or a salicylic acid derivative; (x) a glycerol ester of acetoacetic acid; (xi) a cyclodextrin or cyclodextrin derivative; (xii) a medium-chain or short-chain (e.g. C1 to C 12) fatty acid; and (xiii) a chelating agent; (xiv) an amino acid or salt thereof; and (xv) an N-acetylamino acid or salt thereof.

Solubility enhancers may increase the concentration of the drug or pharmaceutically acceptable salt thereof in the formulation. Useful solubility enhancers include, e.g., alcohols and polyalcohols.

An isotonizing agent may improve the tolerance of the formulation in a nasal cavity. A common isotonizing agent is NaCl. Preferably, when the formulation is an isotonic intranasal dosage formulation, it includes about 0.9% NaCl (v/v) in the aqueous portion of the liquid carrier.

The thickeners may improve the overall viscosity of the composition, preferably to values close to those of the nasal mucosa. Suitable thickeners include methylcellulose, carboxymethylcellulose, polyvinypyrrolidone, sodium alginate, hydroxypropylmethylcellulose, and chitosan.

A humectant or anti-irritant improves the tolerability of the composition in repeated applications. Suitable compounds include, e.g. glycerol, tocopherol, mineral oils, and chitosan.

Various additional ingredients can be used in the compositions of the present invention. The compositions can comprise one or more further ingredients selected from a preservative, an antioxidant, an emulsifier, a surfactant or wetting agent, an emollient, a film-forming agent, or a viscosity modifying agent. These components can be employed and used at levels appropriate for the formulation based on the knowledge of one with ordinary skill in the pharmaceutical and formulation arts. The amounts could range from under 1 percent by weight to up to 90 percent or even over 99 percent by weight.

In one aspect, a preservative can be included. In another aspect, an antioxidant can be included. In another aspect, an emulsifier can be included. In another aspect, an emollient can be included. In another aspect, a viscosity modifying agent can be included. In another aspect, a surfactant or wetting agent can be included. In another aspect, a film forming agent can be included. In another aspect, the pharmaceutical composition is in the form selected from the group consisting of a gel, ointment, lotion, emulsion, cream, liquid, spray, suspension, jelly, foam, mousse, paste, tape, dispersion, aerosol. These components can be employed and used at levels appropriate for the formulation based on the knowledge of one with ordinary skill in the pharmaceutical and formulation arts.

It has surprisingly been found that penetration enhancers such as methyl Beta-cyclodextrin, caprylocaproyl macrogol-8 glycerides, and 2-(2-ethoxyethoxy)ethanol are particularly useful for preparing an intranasal suramin formulation having improved penetration of mucosal tissue.

In another aspect, the at least one preservative can be selected from the group consisting of parabens (including butylparabens, ethylparabens, methylparabens, and propylparabens), acetone sodium bisulfite, alcohol, benzalkonium chloride, benzethonium chloride, benzoic acid, benzyl alcohol, boric acid, bronopol, butylated hydroxyanisole, butylene glycol, calcium acetate, calcium chloride, calcium lactate, cetrimide, cetylpyridinium chloride, chlorhexidine, chlorobutanol, chlorocresol, chloroxylenol, cresol, edetic acid, glycerin, hexetidine, imidurea, isopropyl alcohol, monothioglycerol, pentetic acid, phenol, phenoxyethanol, phenylethyl alcohol, phenylmercuric acetate, phenylmercuric borate, phenylmercuric nitrate, potassium benzoate, potassium metabisulfite, potassium nitrate, potassium sorbate, propionic acid, propyl gallate, propylene glycol, propylparaben sodium, sodium acetate, sodium benzoate, sodium borate, sodium lactate, sodium metabisulfite, sodium propionate, sodium sulfite, sorbic acid, sulfur dioxide, thimerosal, zinc oxide, and N-acetylcysteine, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one antioxidant can be selected from the group consisting of acetone sodium bisulfite, alpha tocopherol, ascorbic acid, ascorbyl palmitate, butylated hydroxyanisole, butylated hydroxytoluene, citric acid monohydrate, dodecyl gallate, erythorbic acid, fumaric acid, malic acid, mannitol, sorbitol, monothioglycerol, octyl gallate, potassium metabisulfite, propionic acid, propyl gallate, sodium ascorbate, sodium formaldehyde sulfoxylate, sodium metabisulfite, sodium sulfite, sodium thiosulfate, sulfur dioxide, thymol, vitamin E polyethylene glycol succinate, and N-acetylcysteine, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one emulsifier can be selected from the group consisting of acacia, agar, ammonium alginate, calcium alginate, carbomer, carboxymethylcellulose sodium, cetostearyl alcohol, cetyl alcohol, cholesterol, diethanolamine, glyceryl monooleate, glyceryl monostearate, hectorite, hydroxypropyl cellulose, hydroxypropyl starch, hypromellose, lanolin, lanolin alcohols, lauric acid, lecithin, linoleic acid, magnesium oxide, medium-chain triglycerides, methylcellulose, mineral oil, monoethanolamine, myristic acid, octyldodecanol, oleic acid, oleyl alcohol, palm oil, palmitic acid, pectin, phospholipids, poloxamer, polycarbophil, polyoxyethylene alkyl ethers, polyoxyethylene castor oil derivatives, polyoxyehtylene sorbitan fatty acid esters, polyoxyethylene stearates, polyoxyl 15 hydroxystearate, polyoxyglycerides, potassium alginate, propylene glycol alginate, propylene glycol dilaurate, propylene glycol monolaurate, saponite, sodium borate, sodium citrate dehydrate, sodium lactate, sodium lauryl sulfate, sodium stearate, sorbitan esters, starch, stearic acid, sucrose stearate, tragacanth, triethanolamine, tromethamine, vitamin E polyethylene glycol succinate, wax, and xanthan gum, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one emollient can be selected from the group consisting of almond oil, aluminum monostearate, butyl stearate, canola oil, castor oil, cetostearyl alcohol, cetyl alcohol, cetyl palmitate, cholesterol, coconut oil, cyclomethicone, decyl oleate, diethyl sebacate, dimethicone, ethylene glycol stearates, glycerin, glyceryl monooleate, glyceryl monostearate, isopropyl isostearate, isopropyl myristate, isopropyl palmitate, lanolin, lanolin alcohols, lecithin, mineral oil, myristyl alcohol, octyldodecanol, oleyl alcohol, palm kernel oil, palm oil, petrolatum, polyoxyethylene sorbitan fatty acid esters, propylene glycol dilaurate, propylene glycol monolaurate, safflower oil, squalene, sunflower oil, tricaprylin, triolein, wax, xylitol, zinc acetate, or a combination thereof. These components can be employed and used at levels appropriate for the formulation based on the knowledge of one with ordinary skill in the pharmaceutical and formulation arts. The amounts could range from under 1 percent by weight to up to 60 percent by weight.

In another aspect, the at least one viscosity modifying agent can be selected from the group consisting of acacia, agar, alginic acid, aluminum monostearate, ammonium alginate, attapulgite, bentonite, calcium alginate, calcium lactate, carbomer, carboxymethylcellulose calcium, carboxymethylcellulose sodium, carrageenan, cellulose, ceratonia, ceresin, cetostearyl alcohol, cetyl palmitate, chitosan, colloidal silicon dioxide, corn syrup solids, cyclomethicone, ethylcellulose, gelatin, glyceryl behenate, guar gum, hectorite, hydrophobic colloidal silica, hydroxyethyl cellulose, hydroxyethylmethyl cellulose, hydroxypropyl cellulose, hydroxypropyl starch, hypromellose, magnesium aluminum silicate, maltodextrin, methylcellulose, myristyl alcohol, octyldodecanol, palm oil, pectin, polycarbophil, polydextrose, polyethylene oxide, polyoxyethylene alkyl ethers, polyvinyl alcohol, potassium alginate, propylene glycol alginate, pullulan, saponite, sodium alginate, starch, sucrose, sugar, sulfoburylether β-cyclodextrin, tragacanth, trehalose, and xanthan gum, or a combination thereof. These components can be employed and used at levels appropriate for the formulation based on the knowledge of one with ordinary skill in the pharmaceutical and formulation arts. The amounts could range from under 1 percent by weight to up to 60 percent.

In another aspect, the at least one film forming agent can be selected from the group consisting of ammonium alginate, chitosan, colophony, copovidone, ethylene glycol and vinyl alcohol grafted copolymer, gelatin, hydroxypropyl cellulose, hypromellose, hypromellose acetate succinate, polymethacrylates, poly(methyl vinyl ether/maleic anhydride), polyvinyl acetate dispersion, polyvinyl acetate phthalate, polyvinyl alcohol, povidone, pullulan, pyroxylin, and shellac, or a combination thereof. These components can be employed and used at levels appropriate for the formulation based on the knowledge of one with ordinary skill in the pharmaceutical and formulation arts. The amounts could range from under 1 percent by weight to up to 90 percent or even over 99 percent by weight.

In another aspect, the at least one surfactant or wetting agent can be selected from the group consisting of docusate sodium, phospholipids, sodium lauryl sulfate, benzalkonium chloride, cetrimide, cetylpyridinium chloride, alpha tocopherol, glyceryl monooleate, myristyl alcohol, poloxamer, polyoxyethylene alkyl ethers, polyoxyethylene castor oil derivatives, polyoxyethylene sorbitan fatty acid esters, polyoxyethylene stearates, polyoxyl 15 hydroxystearate, polyoxyglycerides, propylene glycol dilaurate, propylene glycol monolaurate, sorbitan esters, sucrose stearate, tricaprylin, and vitamin E polyethylene glycol succinate, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, a buffering agent can be included. In another aspect, an emollient can be included. In another aspect, an emulsifying agent can be included. In another aspect, an emulsion stabilizing agent can be included. In another aspect, a gelling agent can be included. In another aspect, a humectant can be included. In another aspect, an ointment base or oleaginous vehicle can be included. In another aspect, a suspending agent can be included. In another aspect an acidulant can be included. In another aspect, an alkalizing agent can be included. In another aspect, a bioadhesive material can be included. In another aspect, a colorant can be included. In another aspect, a microencapsulating agent can be included. In another aspect, a stiffening agent can be included. These components can be employed and used at levels appropriate for the formulation based on the knowledge of one with ordinary skill in the pharmaceutical and formulation arts. The amounts could range from under 1 percent by weight to up to 90 percent or even over 99 by weight.

When the active ingredient is delivered as a powder, the powdered material is often combined with a powdered dispersant. In other embodiments the active can be combined with the dispersant to form particles containing both the active and the dispersant. In yet other embodiments, the active can be coated onto the surface of the dispersant. Examples of dispersants include a wide array of ingredients including sugars, such as lactose, glucose, and sucrose.

One of ordinary skill in the pharmaceutical and formulation arts can determine the appropriate levels of the essential and optional components of the compositions of the present invention.

Methods of preparing the suramin compositions are also intended as part of the present invention and would be apparent to one of ordinary skill in the pharmaceutical and formulation arts using standard formulation and mixing techniques.

Also provided in the present invention is a device for patient administration or self-administration of the antipurinergic agent comprising a nasal spray inhaler containing an aerosol spray formulation of the antipurinergic agent and a pharmaceutically acceptable dispersant or solvent system, wherein the device is designed (or alternatively metered) to disperse an amount of the aerosol formulation by forming a spray that contains the dose of the antipurinergic agent. In other embodiments, the inhaler can comprise the antipurinergic agent as a fine powder, and further in combination with particulate dispersants and diluents, or alternatively combined to form or coat the particulate dispersants.

### EXAMPLES

The following examples further describe and demonstrate embodiments within the scope of the present invention. The Examples are given solely for purpose of illustration and are not to be construed as limitations of the present invention, as many variations thereof are possible without departing from the spirit and scope of the invention.

### Example 1: Composition for Intranasal Delivery

The following composition is prepared using standard mixing equipment and procedures.

| Ingredient | Amount |
|---|---|
| Suramin hexa-sodium salt | 10-200 mg/ml* |
| Methyl beta-cyclodextrin | 40% weight |
| (methyl betadex) | |
| Water | QS to achieve the indicated |
| | levels of ingredients |

*Based on the suramin hexa-sodium salt having a molecular weight of 1429.15 grams/mole

The suramin sodium salt is dissolved in water with gentle mixing. The cyclodextrin is added with mixing until dissolved. The resultant solution is allowed to sit for 2 hours before using.

The composition can be packaged in a spray bottle for intranasal administration.

Alternatively, the compositions are prepared replacing the methyl βeta-cyclodextrin with an equal weight of caprylocaproyl macrogol-8 glycerides or and 2-(2-ethoxyethoxy)ethanol.

The compositions are useful for treating an autism spectrum disorder.

### Example 2: Composition for Intranasal Delivery

The following composition is prepared using standard mixing equipment and procedures.

| Ingredient | Amount |
|---|---|
| Suramin hexa-sodium salt | 10-200 mg/ml* |
| Methyl beta-cyclodextrin | 40% weight |
| (methyl betadex) | |
| Sodium chloride | 0.75% weight |
| Hydroxypropyl methyl cellulose | 0.1% weight |
| Water | QS to achieve the indicated |
| | levels of ingredients |

*Based on the suramin hexa-sodium salt having a molecular weight of 1429.15 grams/mole

The suramin sodium salt is dissolved in water with gentle mixing. The sodium chloride and the hydroxypropyl methyl cellulose are added with mixing. The cyclodextrin is added with mixing until dissolved. The resultant solution is allowed to sit for 2 hours before using.

The composition can be packaged in a spray bottle for intranasal administration.

Alternatively, compositions are prepared replacing the methyl βeta-cyclodextrin with an equal weight of caprylocaproyl macrogol-8 glycerides or and 2-(2-ethoxyethoxy)ethanol.

The compositions are useful for treating an autism spectrum disorder.

### Example 3: Tissue Permeation of Suramin

Four formulations, A-D, were prepared using the methods of Examples 1 and 2 and found to be stable for at least 4 weeks at 25° C. and 60% relative humidity for three months.
Formulation A—suramin hexa-sodium salt at 100 mg/mL in water (no excipients)
Formulation B—suramin hexa-sodium salt at 100 mg/mL in water, with 40% methyl β-cyclodextrin (methyl betadex)
Formulation C—suramin hexa-sodium salt at 100 mg/mL in water, with 40% HP (hydroxyl propyl)-cyclodextrin
Formulation D—suramin hexa-sodium salt at 160 mg/mL in water (no excipients)
   The formulations also contained 0.1% of hydroxypropyl methyl cellulose (i.e. HPMC E5, from Dow Chemicals) as a solution thickening agent; and 0.75% sodium chloride as osmolarity agent.

These four formulations were evaluated in an in vitro permeation study using cultured human airway tissues (EpiAirway AIR-100, purchased from MatTek Corporation), following an established drug permeability protocol (EpiAirway™ Drug Permeation Protocol, MatTek Corporation, 2014). EpiAirway is representative of the upper airways extending from the trachea to the primary bronchi, therefore it is used to measure drug delivery from nasal formulations.

For receiver fluid preparation, one pre-warms the EpiAirway assay medium to 37° C. Using a sterile technique, one pipets 0.3 mL medium into each well of a sterile 24-well plate. Label the wells. Use 0.2 mL of donor solution on the tissues.

Permeability experiment: Following the overnight equilibration, move the cell culture inserts to the 1 hr wells and pipet the donor solution onto the tissue. Return the plates to the incubator. After 30 minutes of elapsed permeation time, move the tissues to 2-hour wells. Similarly move the tissues after 2.0, 3.0, 4.0 and 6.0 hours of total elapsed time. It will not be necessary to replenish the donor solution. Alternatively, one can completely remove the receiver solution at the appropriate time and replace with fresh, pre-warmed receiver fluid. The solutions were analyzed using HPLC and detection at 238 nm.

The following Table 1 provides the averaged accumulated amount, in mg, of suramin that has penetrated as a function of time.

TABLE 1
Total Accumulated Suramin (mg)

| | Formulation | | | |
|---|---|---|---|---|
| Time (hours) | A | B | C | D |
| 1 | 0.047 | 2.629 | 0.000 | 0.082 |
| 2 | 0.145 | 6.011 | 0.055 | 0.249 |
| 3 | 0.258 | 7.276 | 0.171 | 0.436 |
| 4 | 0.391 | 7.969 | 0.386 | 0.692 |
| 5 | 0.773 | 8.863 | 1.443 | 1.278 |
| 6 | 0.047 | 2.629 | 0.000 | 0.082 |

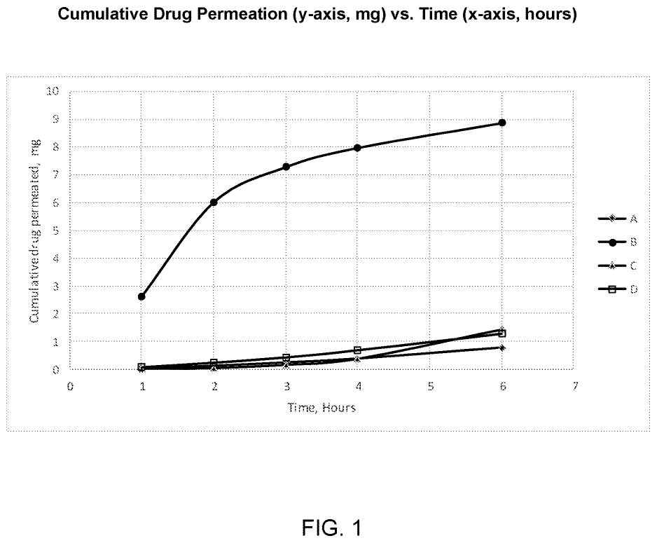

The results of the study are also shown graphically in FIG. 1 where the cumulative amount (mg) of drug permeated was plotted versus time in hours.

These data demonstrate that Formulation B containing methyl β-cyclodextrin (methyl betadex) provides significantly better penetration, versus Formulations, A, C, and D in the tissue permeation assay. Also, as is seen from a comparison of Formulations A and D, having a higher drug concentration can be advantageous to increasing permeation.

### Example 4: Tissue Permeation of Suramin

Six formulations, A-F, were prepared using the methods of Examples 1 and 2 and found to be stable for at least 4 weeks at ambient conditions.
Formulation A—suramin at 200 mg/mL in water (no excipients)
Formulation B—suramin at 140 mg/mL in water, with 40% polysorbate 80 (Tween 80)
Formulation C—suramin at 140 mg/mL in water, with 40% methyl Beta-cyclodextrin (methyl betadex)
Formulation D—suramin at 140 mg/mL in water, with 40% sulfobutylether beta-cyclodextrin (Captisol)
Formulation E—suramin at 140 mg/mL in water, with 40% 2-(2-ethoxyethoxy)ethanol (Transcutol P)
Formulation F—suramin at 140 mg/mL in water (Labrasol)

Tissue permeability studies were conducted using the methods of Example 3.

The following Table 2 provides the averaged accumulated amount, in mg, of suramin that has penetrated as a function of time.

TABLE 2

| | Total Accumulated Suramin (mg) | | | | | |
|---|---|---|---|---|---|---|
| Time | Formulation | | | | | |
| (hours) | A | B | C | D | E | F |
| 1 | 0.09 | 0.05 | 3.69 | 0.05 | 1.47 | 3.20 |
| 2 | 0.40 | 0.39 | 12.22 | 0.45 | 5.03 | 6.77 |
| 3 | 1.01 | 0.65 | 15.57 | 1.12 | 8.67 | 8.23 |
| 4 | 2.16 | 1.08 | 19.11 | 2.41 | 13.32 | 9.74 |
| 6 | 5.93 | 1.88 | 22.24 | 5.63 | 17.90 | 13.17 |

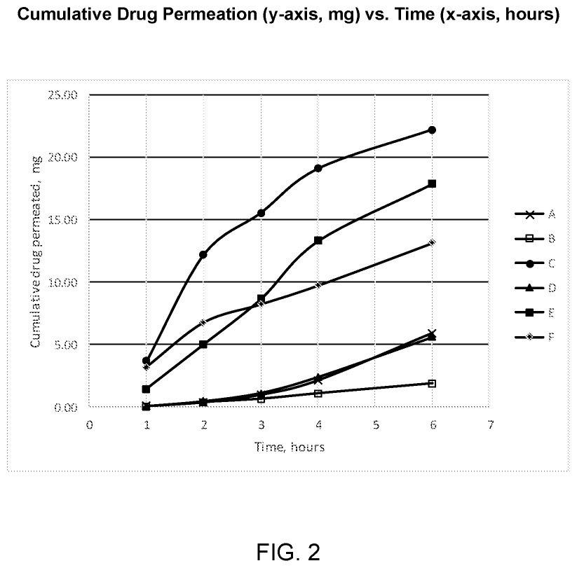

The results of the study are also showing graphically in FIG. 2 where the cumulative amount (mg) of drug permeated was plotted versus time in hours. These data demonstrate that Formulation C containing methyl Beta-cyclodextrin (methyl betadex), E containing 2-(2-ethoxyethoxy)ethanol (Transcutol P), and F containing caprylocaproyl macrogol-8 glycerides (Labrasol) provide significantly better penetration, versus Formulations, A, B, and D in the tissue permeation assay.

Furthermore, the results from Examples 3 and 4 are surprising.

Cyclodextrins are sugar molecules bound together in rings of various sizes. Specifically, the sugar units are called glucopyranosides—glucose molecules that exist in the pyranose (six-membered) ring configuration. Six, 8, or 10 glucopyranosides bind with each other to form α-, β-, and γ-cyclodextrin, respectively. Cyclodextrins form a toroid (truncated cone) configuration with multiple hydroxyl groups at each end. This allows them to encapsulate hydrophobic compounds without losing their solubility in water. Among other applications, cyclodextrins can be used to carry hydrophobic drug molecules into biological systems, as tissue permeation enhancers. It has been reported that the cyclodextrins form inclusion complexes with a variety of hydrophobic drugs thereby increasing their partitioning and solubility in the tissue membrane. Methyl Beta-cyclodextrin (betadex) is a type of cyclodextrin. Methyl betadex is used in at least one marketed intranasal product Estradiol (Aerodiol) to enhance trans-tissue permeation of the drug molecule, estradiol (MW=272.4). Because of its small size (MW=272.4), estradiol molecule can be easily encapsulated into the cyclodextrin ring, and thus enhancement of delivery into biological tissues is achieved.

However, we discovered a way in which methyl Beta-cyclodextrin could also be capable of encapsulating suramin, which is a much larger molecule than generally considered compatible. It is surprising to find the methyl betadex works for suramin. A person having ordinary skill in the art would not have been expected that such a large molecule could be encapsulated into cyclodextrin ring.

Another useful penetration enhancer is Transcutol P (Diethylene glycol mono-ethyl ether). This is an excipient which has been reported to enhancer skin permeability for some small molecule drug compounds in various topical/transdermal formulations. Nevertheless, it has not been used as an excipient for intranasal products. Also, it is not commonly used to enhance large molecule such as suramin.

Another useful penetration enhancer is Labrasol (Caprylocaproyl macrogol-8 glycerides). This is an excipient that have been reported to enhancer skin permeability of some drug compounds in some topical/transdermal formulations. It has not been used as an excipient for intranasal products.

### Example 5: Determination of Suramin in Plasma and Brain Tissue

The following example describes a mouse study conducted to determine the delivery of suramin to plasma and brain tissue when administered intraperitoneally (IP) or intranasally (IN) according to different treatment regimens. For the study, male Fmr1-knockout B6.129P2-Fmr1tm1Cgr/J TG mice were purchased from Jackson Laboratories, Bar Harbor, Me. These mice were of approximately 8 weeks of age. These mice exhibit abnormalities of dendritic spines in multiple regions of the brain. The absence of FMRP in these mice induces an over-activation of RAC1, a protein of the Rho GTPase subfamily that plays a critical role in dendritic morphology and synaptic function. These B6.129P2-Fmr1tm1Cgr/J TG mice, provide an animal model for cognitive disabilities and neurodevelopmental disorders.

The mice were maintained in group cages (6 mice per cage based on treatment group) in a controlled environment (temperature: 21.5±4.5° C. and relative humidity: 35-55%) under a standard 12-hour light/12-hour dark lighting cycle (lights on at 06:00). Mice were accommodated to the research facility for approximately a week. Body weights of all mice were recorded for health monitoring purposes.

The mice were divided into the following 5 test groups, with 6 mice per group.
Group 1: Intraperitoneal (IP) injection of suramin, 20 mg/kg, administered weekly to animals beginning at 9 weeks of age and continuing for four weeks (i.e. given at Age Weeks 9, 10, 11 and 12). The suramin was formulated in Normal saline solution.
Group 2: Intraperitoneal (IP) injection of saline, 5 mL/g, administered weekly to animals beginning at 9 weeks of age and continuing for four weeks (i.e. given at Age Weeks 9, 10, 11 and 12). This was a control group.
Group 3: Intranasal (IN) administration of a formulation, described below, of suramin, at a concentration of 100 mg/mL×6 mL per spray, administered as one spray per nostril, one time per day, (interval of each application is around 2 minutes to ensure absorption) for 28 days (total of 56 sprays over 28 day period) beginning at 9 weeks of age (i.e. given daily during Age Weeks 9, 10, 11 and 12).
Group 4: Intranasal (IN) administration of a formulation, described below, of suramin, at a concentration of 100 mg/mL×6 mL per spray, administered as one spray per nostril, one time every other day, for 28 days (total of 28 sprays over 28 day period) beginning at 9 weeks of age (i.e. given once every other day during Age Weeks 9, 10, 11 and 12).
Group 5: Intranasal (IN) administration of a formulation, described below, of suramin, at a concentration of 100 mg/mL×6 mL per spray, administered as one spray per nostril, one time every week, for 4 weeks (28 days) (total of 8 sprays over 28 day period) beginning at 9 weeks of age (i.e. given once weekly during Age Weeks 9, 10, 11 and 12).

The following is the suramin intranasal (IN) formulation administered to Groups 3, 4, and 5, above.

| | Weight (grams) | Percent of Composition |
|---|---|---|
| Suramin hexa-sodium salt | 16.6 | 10.3% |
| Methyl beta cyclodextrin | 50 | 30.9% |
| Benzalkonium chloride | 0.04 | 0.012% |
| (50% aqueous solution) | | |
| HPMC E5* | 5.6 | 3.46% |
| Citric acid | 0.3 | 0.19% |
| Sodium sulfite | 0.15 | 0.093% |
| Water | 89.13 | 55.1% |
| Total | 161.82 | 100% |

*HPMC E5 is a water-soluble cellulose ethers polymer [hydroxypropyl methylcellulose (HPMC)] available from DuPont.

The above formulation is made by dissolving the suramin sodium salt in water with gentle mixing. The remaining ingredients, except the cyclodextrin are added with mixing. The cyclodextrin is then added with mixing until dissolved. The resultant solution is allowed to sit for 2 hours before using.

Blood samples were collected from all mice at the end of 12 weeks of age. Brain tissue was harvested from all mice upon sacrifice 13-14 weeks of age. Standard sample preparation and analytical techniques were used to obtain the data.

The results from this study are shown in Table 3. The data is presented as the average plasma concentration (in both ng/ml and μM) for each animal group the and average brain tissue concentration (in both ng/g and mmol/g). Also presented is the average brain tissue to plasma partition ratio for each group. Note that such a calculation is not applicable for the group administered a saline control (Group 2) as no suramin was detected in the brain tissue and the small plasma levels are essentially noise from the analytical method.

TABLE 3

| | Average Plasma | | Average Brain Tissue | | Average Brain Tissue |
|---|---|---|---|---|---|
| | Concentration | | Concentration | | to Plasma |
| Group | ng/ml | μM | ng/g | mmol/g | Partitioning Ratio¹ |
| 1 | 18733 | 14.440 | 550 | 0.424 | 0.030 |
| 2 | 88.3 | 0.068 | BQL² | BQL² | NA³ |
| 3 | 1637 | 1.262 | 115.2 | 0.089 | 0.069 |
| 4 | 1578 | 1.217 | 127.5 | 0.098 | 0.089 |
| 5 | 278.7 | 0.215 | 91.3 | 0.070 | 0.235 |

¹BQL means below quantifiable limit.
²NA means not applicable.
³The partitioning ratio is calculated directly from the raw data rather than the averages presented in the table.

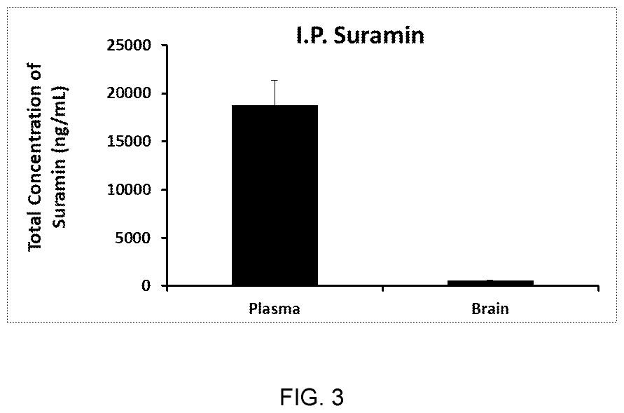

The results from the study are also shown in the plots of FIGS. 3 through 10.

FIG. 3 shows a plot of the total concentration, in ng/ml, of suramin in plasma versus brain tissue in mice when administered by intraperitoneal (IP) injection, 20 mg/kg, weekly to the mice beginning at 9 weeks of age and continuing for four weeks (i.e. given at age weeks 9, 10, 11 and 12).

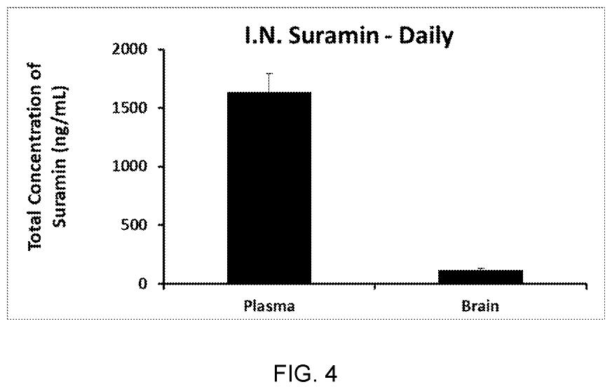

FIG. 4 shows a plot comparing the total concentration, in ng/ml, of suramin in plasma versus brain tissue in mice when administered intranasally (IN) daily for 28 days. A composition of the present invention comprising IN suramin, at a concentration of 100 mg/mL×6 mL per spray, was administered as one spray per nostril, one time per day, (interval of each application is around 2 minutes to ensure absorption) for 28 days (total of 56 sprays over 28 day period) beginning at 9 weeks of age (i.e. given daily during age weeks 9, 10, 11 and 12).

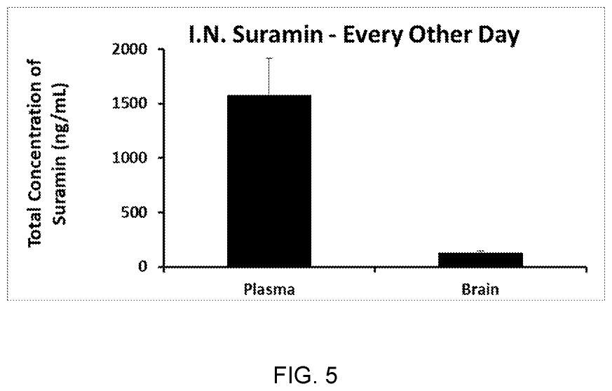

FIG. 5 shows a plot comparing the total concentration, in ng/ml, of suramin in plasma versus brain tissue in mice when administered intranasally (IN) every other day for 28 days. A composition of the present invention comprising IN suramin, at a concentration of 100 mg/mL×6 mL per spray, was administered as one spray per nostril, every other day, (interval of each application is around 2 minutes to ensure absorption) for 28 days (total of 28 sprays over 28 day period) beginning at 9 weeks of age (i.e. given daily during age weeks 9, 10, 11 and 12).

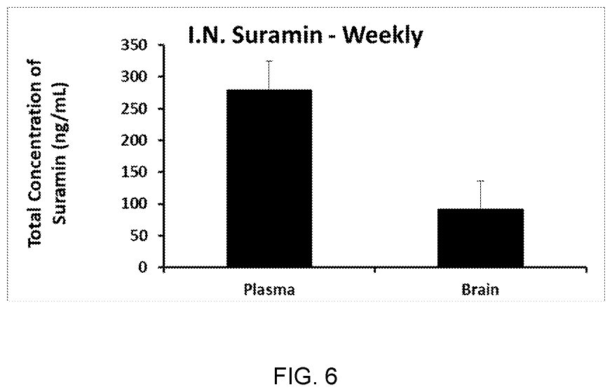

FIG. 6 shows a plot comparing the total concentration, in ng/ml, of suramin in plasma versus brain tissue in mice when administered intranasally (IN) once per week for 4 weeks. A composition of the present invention comprising IN suramin, at a concentration of 100 mg/mL×6 mL per spray, was administered as one spray per nostril, one time per week, (interval of each application is around 2 minutes to ensure absorption) for 4 weeks (28 days) (total of 8 sprays over 28 day period) beginning at 9 weeks of age (i.e. given daily during age weeks 9, 10, 11 and 12).

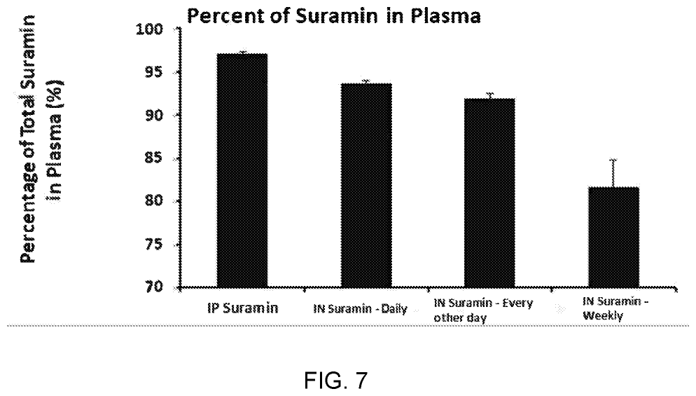

FIG. 7 shows a plot comparing the total percentage of suramin in plasma in mice when administered by intraperitoneal (IP) injection once weekly for 4 weeks (28 days), intranasally (IN) daily for 28 days, intranasally (IN) every other day for 28 days, and intranasally (IN) once per week for 4 weeks (28 days).

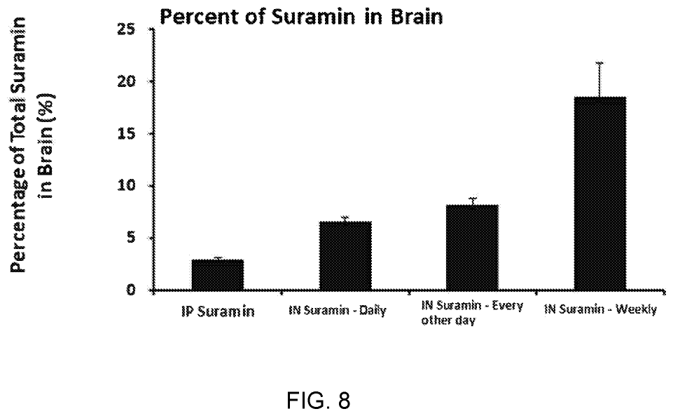

FIG. 8 shows a plot comparing the total percentage of suramin in brain tissue in mice when administered by intraperitoneal (IP) injection once weekly for 4 weeks (28 days), intranasally (IN) daily for 28 days, intranasally (IN) every other day for 28 days, and intranasally (IN) once per week for 4 weeks (28 days).

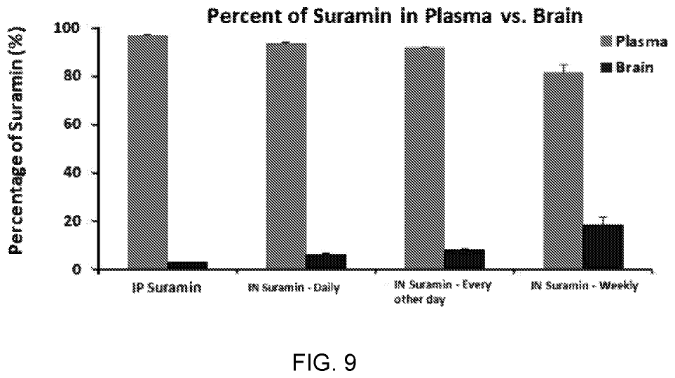

FIG. 9 shows a plot comparing the total percentage of suramin in plasma versus brain tissue in mice when administered by intraperitoneal (IP) injection once weekly for 4 weeks (28 days), intranasally (IN) daily for 28 days, intranasally (IN) every other day for 28 days, and intranasally (IN) once per week for 4 weeks (28 days).

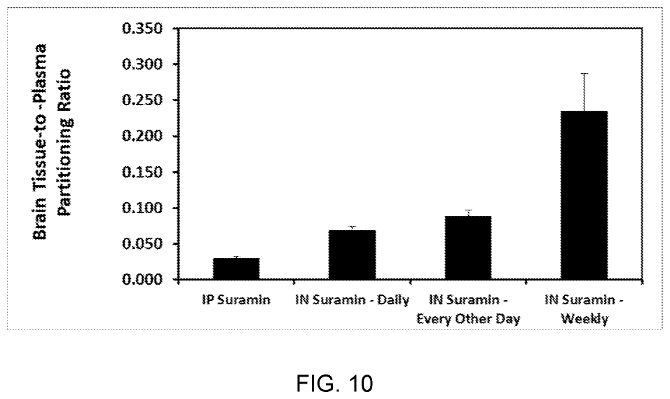

FIG. 10 shows a plot comparing the brain tissue to plasma partitioning ration of suramin in mice when administered by intraperitoneal (IP) injection once weekly for 4 weeks (28 days), intranasally (IN) daily for 28 days, intranasally (IN) every other day for 28 days, and intranasally (IN) once per week for 4 weeks (28 days).

These results demonstrate that an antipurinergic agent such as suramin can be delivered intranasally to achieve plasma and brain tissue levels and that variations in the brain tissue to plasma partitioning ratio can be observed. These results demonstrate that an antipurinergic agent such as suramin can be delivered to the brain of a mammal by intranasal (IN) administration.

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the methods and compositions of the present invention, where the term comprises is used with respect to the recited steps of the methods or components of the compositions, it is also contemplated that the methods and compositions consist essentially of, or consist of, the recited steps or components. Furthermore, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

Furthermore, it should be recognized that in certain instances a composition can be described as being composed of the components prior to mixing, because upon mixing certain components can further react or be transformed into additional materials.

All percentages and ratios used herein, unless otherwise indicated, are by weight. It is recognized the mass of an object is often referred to as its weight in everyday usage and for most common scientific purposes, but that mass technically refers to the amount of matter of an object, whereas weight refers to the force experienced by an object due to gravity. Also, in common usage the “weight” (mass) of an object is what one determines when one “weighs” (masses) an object on a scale or balance.

1. A method for treating cognitive, social, or behavioral disabilities and neurodevelopmental disorders, comprising intranasally administering a therapeutically effective amount of a pharmaceutical composition comprising a therapeutically effective amount of an antipurinergic agent, or a pharmaceutically acceptable salt, ester, solvate, or prodrug thereof to a patient in need thereof.

2. The method of claim 1 wherein the patient is a human.

3. The method of claim 1 wherein the cognitive, social, behavioral disability, or neurodevelopmental disorder is selected from the group consisting of autism spectrum disorder, FXS, FXTAS, CFS, and PTSD.

4-8. (canceled)

9. The method of claim 3 wherein said autism spectrum disorder is selected from the group consisting of autistic disorder, childhood disintegrative disorder, pervasive developmental disorder-not otherwise specified (PDD-NOS), and Asperger syndrome, and wherein said autism spectrum disorder includes one or more symptoms selected from the group consisting of difficulty communicating, difficulty interacting with others, and repetitive behaviors.

10. (canceled)

11. The method of claim 1 wherein said antipurinergic agent is suramin, or a pharmaceutically acceptable salt, ester, solvate, or prodrug thereof.

12-13. (canceled)

14. The method of claim 11 wherein said salt is the hexa-sodium salt.

15-16. (canceled)

17. The method of claim 14 wherein said composition further comprises a penetration enhancer.

18. The method of claim 17 wherein said penetration enhancer is selected from the group consisting of methyl Beta-cyclodextrin, caprylocaproyl macrogol-8 glycerides, 2-(2-ethoxyethoxy)ethanol, and combinations thereof.

19-21. (canceled)

22. The method of claim 1 wherein said composition is administered at least once daily, or at least twice daily, or at least once weekly, or at least twice weekly, or at least once biweekly (i.e. every two weeks), or at least once monthly, or at least once every 4 weeks.

23. The method of claim 1 wherein said composition administered at least once about every 41 days to about 78 days.

24-25. (canceled)

26. The method of claim 1 wherein the plasma level of the suramin in the patient is maintained at less than about 3 micromolar (μM), or less than about 2.75 micromolar, or less than about 2.5 micromolar, or less than about 2 micromolar, or less than about 1 micromolar, or less than about 0.5 micromolar based on the suramin active.

27. The method of claim 1 wherein the brain tissue level of the suramin in the patient is from about 1 ng/ml to about 1000 ng/ml.

28. (canceled)

29. The method of claim 1 wherein the brain tissue to blood plasma partitioning ratio for the suramin is at least about 0.05, or at least about 0.1, or at least about 0.25, or at least about 0.50.

30. The method of claim 1 wherein the composition comprises from about 0.01 mg to about 200 mg per unit dosage of suramin, based on the suramin active.

31-37. (canceled)

38. The method of claim 1 wherein the AUC for the plasma level for the suramin active for the patient is less than about 80 μg*day/L or is less than about 75 μg*day/L, or is less than about 50 μg*day/L, or is less than about 25 μg*day/L, or is less than about 10 μg*day/L.

39. The method of claim 1 wherein the Cₘₐₓ for the plasma level for the suramin active for the patient is less than about 75 micromolar, or is less than about 7.5 micromolar, or is less than about 0.1 micromolar, and optionally at least about 0.01 micromolar, based on a single dose.

40. The method of claim 1 wherein said composition is in the form of a nasal spray for intranasal administration.

41-42. (canceled)

43. The method of claim 1 wherein the composition exhibits a penetration rate of about 1 micrograms/cm² per hour to about 200 micrograms/cm² per hour of suramin, based on the suramin active, through cultured human airway tissue.

44-47. (canceled)

48. The method of claim 1 wherein treating said autism spectrum disorder, FXS, or FXTAS comprises improving by 10% or more an assessment score of said patient relative to a score from said patient prior to said administration.

49. (canceled)

50. The method of claim 48 wherein the assessment score is selected from ABC, ADOS, ATEC, CARS CGI, and SRS.

51-64. (canceled)

65. A device for patient administration, including administration selected from self-administration and administration to the patient by an individual other than the patient, for treating cognitive, social, or behavioral disabilities and neurodevelopmental disorders according to claim 1, comprising a nasal spray inhaler for intranasally administering a composition comprising an antipurinergic agent.

66. (canceled)
